(19) United States
(12) Patent Application Publication
(10) Pub. No.: US 2025/0316327 A1
(43) Pub. Date: Oct. 9, 2025

(54) STOCHASTIC FLOW MATCHING FOR PROTEIN BACKBONE GENERATION

(71) Applicant: Wise Algorithms Inc., Montreal (CA)

(72) Inventors: Avishek Bose, Mississauga (CA); Tara Akhound-Sadegh, Montreal (CA); Kilian Fatras, Montreal (CA); Guillaume Huguet, Montreal (CA); Jarrid Rector-Brooks, Montreal (CA); Chenghao Liu, Montreal (CA); Pablo Lemos, Mississauga (CA); Maksym Korablyov, Montreal (CA); Alexander Yi-Ren Tong, Montreal (CA); James Vuckovic, Toronto (CA); Eric Thibodeau-Laufer, Montreal (CA)

(21) Appl. No.: 19/017,077

(22) Filed: Jan. 10, 2025

Related U.S. Application Data
(60) Provisional application No. 63/619,535, filed on Jan. 10, 2024.
(60) Provisional application No. 63/653,395, filed on May 30, 2024.

(51) Int. Cl. G16B 15/20; G16B 30/00; G16B 40/20

(52) U.S. Cl. CPC G16B 15/20; G16B 30/00; G16B 40/20

(57) ABSTRACT

Embodiments of the disclosure include the implementation of generative models exhibiting increased modeling power based on flow-matching paradigm over 3D rigid motions. Altogether, these models enable more accurate modeling of protein backbones.

### CROSS-REFERENCE TO RELATED APPLICATIONS

This application claims the benefit of and priority to U.S. Provisional Patent Application No. 63/619,535 filed Jan. 10, 2024, and to U.S. Provisional Patent Application No. 63/653,395 filed May 30, 2024, the entire disclosure of each of which is hereby incorporated by reference in its entirety for all purposes.

### BACKGROUND OF THE INVENTION

Proteins are one of the basic building blocks of life. Their complex geometric structure enables specific inter-molecular interactions that allow for crucial functions within organisms, such as acting as catalysts in chemical reactions, transporters for molecules, and providing immune responses. Normally, such functions arise as a result of evolution. With the emergence of computational techniques, it has become possible to rationally design novel proteins with desired structures that program their functions. Such methods are now seen as the future of drug design and can lead to solutions to long-standing global health challenges.

In protein engineering, the term de novo design refers to a setting when a new protein is designed to satisfy pre-specified structural and functional properties (Huang et al., 2016). Chemically, a protein is a sequence of amino acids (residues) linked into a chain that folds into a complex 3D structure under the influence of electrostatic forces. The protein backbone can be seen as N rigid bodies (corresponding to N residues) that contain four heavy atoms N—C_{α}—C—O. Mathematically, each residue can be associated with a frame that adheres to the symmetries of orientation-preserving rigid transformations (3D rotations and translations), forming the special Euclidean group SE(3) (Jumper et al., 2021); the entire protein backbone is described by the group product SE(3)^{N}. The problem of protein design can be formulated as sampling from the distribution over this group. Recently, generative models have been generalized to Riemannian manifolds (Mathieu & Nickel, 2020; De Bortoli et al., 2022). However, they are not purpose-built to exploit the rich geometric structure of SE(3)^{N}. Furthermore, several approaches require numerically expensive steps like simulating a Stochastic Differential Equation (SDE) during training or using the Riemannian divergence in the objective (Huang et al., 2022; Leach et al., 2022; Ben-Hamu et al., 2022).

### SUMMARY

Disclosed herein are generative machine learning models, also referred to herein as FOLDFLOW. These models represent a family of continuous normalizing flows (CNFs) tailored for distributions on SE(3)^{N}. Here, the models are generated using a framework of Conditional Flow Matching (CFM), a simulation-free approach to learning CNFs by directly regressing time-dependent vector fields that generate probability paths. In particular, disclosed herein are three new CFM-based models that learn SE(3)^{N}-invariant distributions to generate protein backbones. In contrast to the previous SE(3) diffusion approach of Yim et al. (2023b), FOLDFLOW is able to start from an informative prior. This enables new applications of generative models for protein design such as equilibrium conformation generation (Zheng et al., 2023).

A first model, referred to as “FOLDFLOW-BASE” extends the Riemannian flow matching approach (Chen & Lipman, 2023) by introducing a closed-form expression of the ground truth conditional vector field for SO(3) in the loss computation, thus greatly increasing speed and stability of training. Next, for a second model referred to “FOLDFLOW-OT”, the training of the base model is accelerated by constructing straighter and simpler flows using Riemannian Optimal Transport (OT) on SE(3)^{N}. This proves the existence of a Monge map on SE(3)^{N} allowing definition of the OT displacement interpolants. Finally, a third model is a complex simulation-free model referred to as “FOLDFLOW-SFM”, which builds a stochastic bridge that can map arbitrary distributions on SE(3)^{N}. The main contributions are summarized below:
Three new simulation-free generative models in FOLDFLOW-BASE, FOLDFLOW-OT, and FOLDFLOW-SFM that map between any source and target distribution on SE(3)^{N}.
Prove the existence of a Monge map on an invariant subgroup SE(3)NO and build the Riemannian OT problem on

$$SE {( 3 )}_{0}^{N}$$

which power FOLDFLOW-OT, resulting in more stable flows.
Prove FOLDFLOW-SFM is a valid flow and use it to construct a simulation-free SDE on SE(3).
Provide several new numerical techniques, such as a closed-form expression of the target conditional vector field for SO(3), which leads to fast, stable, and efficient training on SO(3).
Empirically validate the proposed models by learning SE(3)^{N}-invariant distributions for protein backbones. All three of the models outperform the current SOTA non-pretrained model in FrameDiff-Improved (Yim et al., 2023b) in in-silico designability, diversity, and novelty.

Additionally disclosed herein is FoldFlow++, a novel sequence-conditioned SE(3)-equivariant flow matching model for protein structure generation. FOLDFLOW++ presents additional architectural features over the previous FOLDFLOW family of models including a protein large language model to encode sequence, a new multi-modal fusion trunk that combines structure and sequence representations, and a geometric transformer based decoder. To increase diversity and novelty of generated samples FOLDFLOW++ is trained at scale on a new dataset that is an order of magnitude larger than PDB datasets of prior works, containing both known proteins in PDB and high-quality synthetic structures achieved through filtering. FOLDFLOW++ was aligned to arbitrary rewards, e.g. increasing secondary structures diversity, by introducing a Reinforced Finetuning (ReFT) objective. Furthermore, FOLDFLOW++ outperformed previous state-of-the-art protein structure-based generative models, improving over RFDiffusion in terms of unconditional generation across all metrics including designability, diversity, and novelty across all protein lengths, as well as exhibiting generalization on the task of equilibrium conformation sampling. Finally, a fine-tuned FOLDFLOW++ made progress on challenging conditional design tasks such as designing scaffolds for the VHH nanobody.

### BRIEF DESCRIPTION OF THE DRAWINGS

These and other features, aspects, and advantages of the present invention will become better understood with regard to the following description and accompanying drawings. It is noted that wherever practicable similar or like reference numbers may be used in the figures and may indicate similar or like functionality. For example, a letter after a reference numeral, such as “third party entity 120A,” indicates that the text refers specifically to the element having that particular reference numeral. A reference numeral in the text without a following letter, such as “third party entity 120,” refers to any or all of the elements in the figures bearing that reference numeral (e.g. “third party entity 120” in the text refers to reference numerals “third party entity 120A” and/or “third party entity 120B” in the figures).

FIG. 1A depicts an example system overview, in accordance with an embodiment.

FIG. 1B depicts a flow diagram for deploying a machine learning model to predict a protein backbone structure, in accordance with an embodiment.

FIG. 1C depicts a flow diagram for training a machine learning model, in accordance with an embodiment.

FIG. 2A depicts a block diagram including a generative predictive model for predicting a protein backbone structure, in accordance with an embodiment.

FIG. 2B depicts a flow diagram for implementing a generative predictive model, in accordance with the embodiment shown in FIG. 2A.

FIG. 3 illustrates an example computing device 300 for implementing system and methods described in FIGS. 1A-1C.

FIGS. 4A-4C show conditional probability paths learned by FOLDFLOW-BASE (FIG. 4A), FOLDFLOW-OT (FIG. 4B), and FOLDFLOW-SFM (FIG. 4C). The rotation trajectory of a single residue is visualized by the action of SO(3) on its homogenous space S2.

FIG. 4D shows properties of each model, properties including: whether they can map from a general source distribution, perform optimal transport, are stochastic, or require the score of the IGSO(3) density.

FIG. 5 depicts an example parameterization of a protein backbone.

FIG. 6 depicts FOLDFLOW-SFM generated structures in green compared to ProteinMPNN→ESMFold refolded structures in grey. Samples with RMSD <2A for lengths 100, 150, 200, 250, 300 from left to right.

FIG. 7A depicts norm of the rotation flow with t scaling.

FIG. 7B depicts scRMSD of designed proteins vs. ESMFold under flow scaling.

FIG. 7C shows an ablation study of FOLDFLOW features against designability.

FIG. 8A shows a Ramachandran plot of Φ and ψ of the most flexible residue (56) in BPTI.

FIG. 8B shows an ICA of all dihedral angles of BPTI.

FIG. 8C shows 1000 BPTI conformations sampled by FOLDFLOW with Ca alignment highlighted in yellow and AlphaFold2 samples in green. FOLDFLOW reproduces test MD frames while AlphaFold2 samples do not.

FIGS. 9A-9C show numerical comparisons between the simulation-free of the SDE in FOLDFLOW-SFM vs. simulated Brownian bridge on SO(3), for different values of the diffusion coefficient, γ.

FIGS. 10A-10D show data visualizations using the Euler-angle representation of the rotation matrices. Data visualizations are from data distribution (FIG. 10A), FOLDFLOW-BASE(FIG. 10B), FOLDFLOW-OT (FIG. 10C), and FOLDFLOW-SFM (FIG. 10D).

FIGS. 11A-11C depicts protein characterizations including designability as quantified by scRMSD (lower is better) (FIG. 11A), diversity as quantified by average pairwise TMScore (lower is better) (FIG. 11B), and novelty of proteins as quantified by maximum TMScore to PDB (lower is better) (FIG. 11C), designed across lengths for FOLDFLOW models and previous state of the art models.

FIGS. 12A-12B show KL divergence per residue of the 2D dihedral angle (@ and) distributions between the samples from FOLDFLOW and test MD frames (blue) and AlphaFold 2 and the test MD frames (orange).

FIG. 13 depicts five proteins generated by FOLDFLOW-SFM from each backbone length.

FIG. 14A shows distribution of protein lengths in the dataset.

FIG. 14B shows distribution of protein lengths in a batch (when sampling uniformly by cluster)

FIG. 14C shows distribution of number of proteins per cluster.

FIGS. 15A-15B shows the FOLDFLOW++ architecture which processes sequence and structure and outputs SE(3)^{N} vectorfields.

FIG. 16 shows designable samples from various methods. Overlayed in silver are refolded ESMFold structures. FOLDFLOW++ exhibits significantly more diversity in secondary structure at shorter lengths than RFDiffusion with fine-tuned models able to produce diverse proteins across lengths.

FIG. 17 shows uncurated designable (scRMSD <2 Å) length 100 structures with ESMFold refolded structure from FOLDFLOW++ and RFDiffusion colored by secondary structure assignment. FOLDFLOW++ is significantly more diverse in terms of secondary structure composition where we see RFDiffusion generates mostly α-helices.

FIGS. 18A-18E show distribution of secondary structure elements (α-helices, β-sheets, and coils) of designable (scRMSD <2.0) proteins generated by various models. FOLDFLOW++ generates more diverse designable backbones.

FIGS. 19A-19D show secondary structure elements distributions (α-helices, β-sheets, and coils) of designable (scRMSD <2.0) proteins, along with the data's distribution. We find more steps leads to more diverse designs with fewer α-helix only generations.

### DETAILED DESCRIPTION OF THE INVENTION

### Definitions

It must be noted that, as used in the specification and the appended claims, the singular forms “a,” “an” and “the” include plural referents unless the context clearly dictates otherwise.

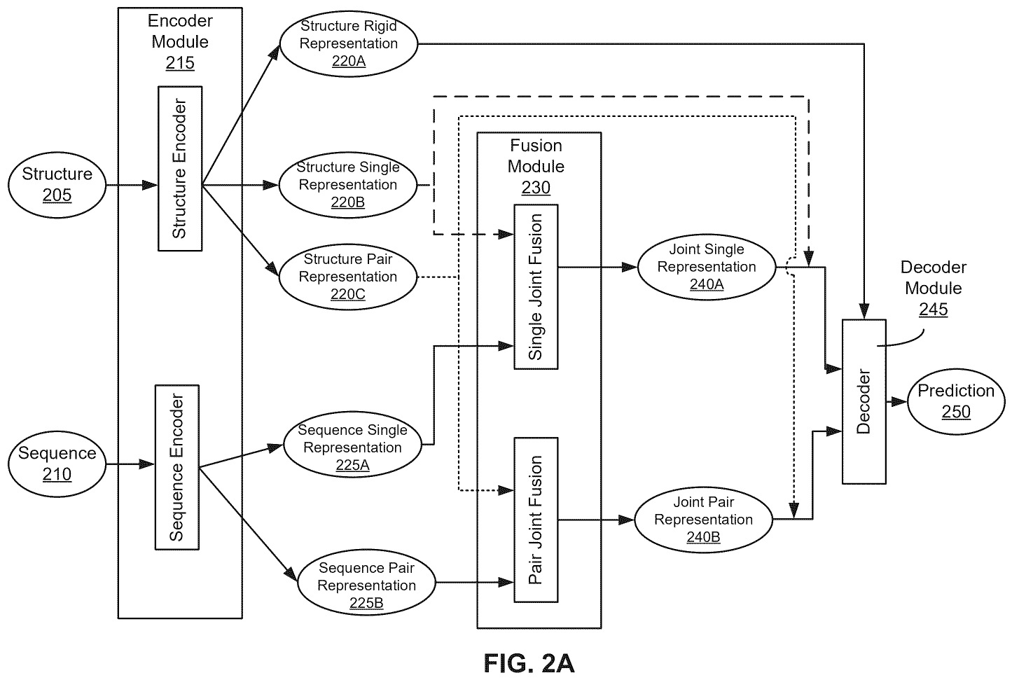

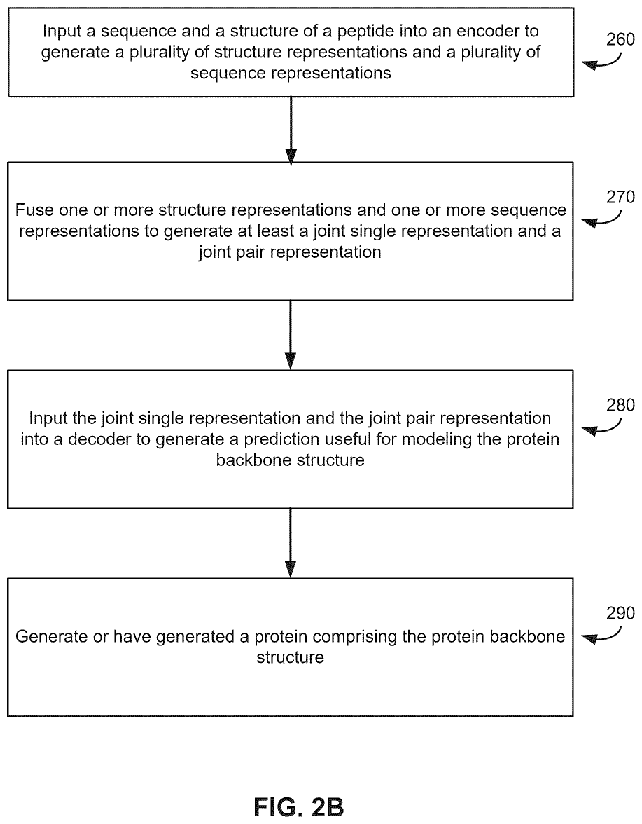

As used herein, the phrase “generative predictive model” refers to a model that analyzes a sequence and a structure of a protein and generates a prediction, such as a prediction useful for useful for modeling a protein backbone structure or a prediction of a protein backbone structure. In various embodiments, a generative predictive model can include a particular model architecture including one or more components. For example, a generative model can include an autoencoder (e.g., an encoder and a decoder). As another example, a generative model can include a multi-modal fusion trunk for fusing representations. In particular embodiments, a generative model includes an encoder that feeds into a multi-modal fusion trunk, which then feeds into the decoder e.g., as shown in FIG. 2A.

As used herein, the term “protein” refers to a compound comprised of at least two amino acid residues covalently linked by peptide bonds or modified peptide bonds, for example peptide isosteres (modified peptide bonds). In various embodiments, a protein includes 2 or more amino acids, 3 or more amino acids, 4 or more amino acids, 5 or more amino acids, 10 or more amino acids, 15 or more amino acids, 20 or more amino acids, 25 or more amino acids, 30 or more amino acids, 40 or more amino acids, 50 or more amino acids, 60 or more amino acids, 70 or more amino acids, 80 or more amino acids, 90 or more amino acids, 100 or more amino acids, 125 or more amino acids, 150 or more amino acids, 175 or more amino acids, 200 or more amino acids, 250 or more amino acids, 300 or more amino acids, 400 or more amino acids, 500 or more amino acids, 600 or more amino acids, 700 or more amino acids, 800 or more amino acids, 900 or more amino acids, or 1000 or more amino acids. In particular embodiments, a protein includes 2, 3, 4, 5, 6, 7, 8, 9, 10, 11, 12, 13, 14, 15, 16, 17, 18, 19, 20, 25, 30, 35, 40, 45, 50, 55, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, or 1000 amino acids.

### Overview

Disclosed herein are exemplary generative predictive models for predicting protein backbone structures. As described in further detail herein, exemplary generative predictive models combine the parametrization of protein backbones and sequences into sequence representations, structure representations, and a rigid representation (e.g., a special Euclidean group SE(3) representing a group of rigid body motions or transformations in three-dimensional space). A fusion trunk of the generative predictive model fuses the representations and a decoder of the generative predictive model consumes the fused representations and outputs a predicted structure (e.g., a protein backbone structure). The methods disclosed herein are useful for enabling downstream applications, including but not limited to:
Synthetic biology applications for enzyme or pathway engineering,
Vaccine development by designing immunogenic epitopes with structural constraints,
Computational de novo protein design for novel therapeutic proteins, and
Optimization of existing protein designs for enhanced stability or activity.
   Additionally, methods disclosed herein, including the methods for implementing the generative predictive model, can be useful as a step in an automated pipeline e.g., for high-throughput protein design and validation.

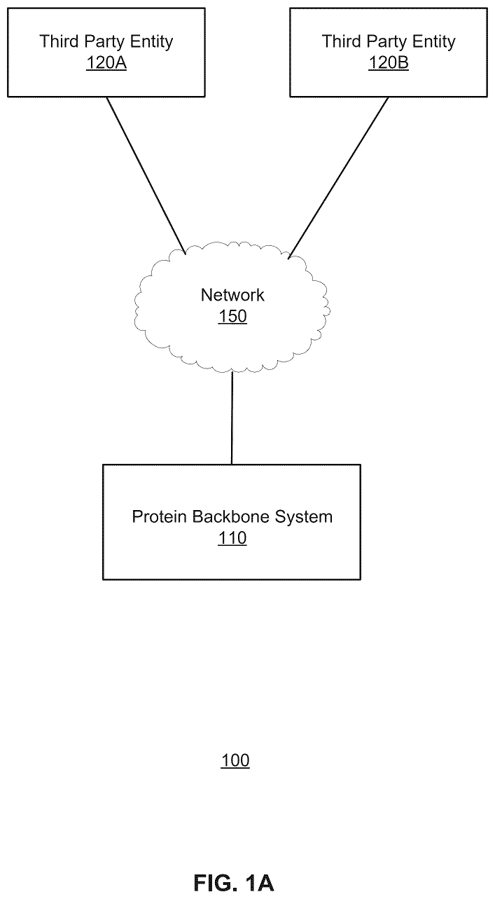

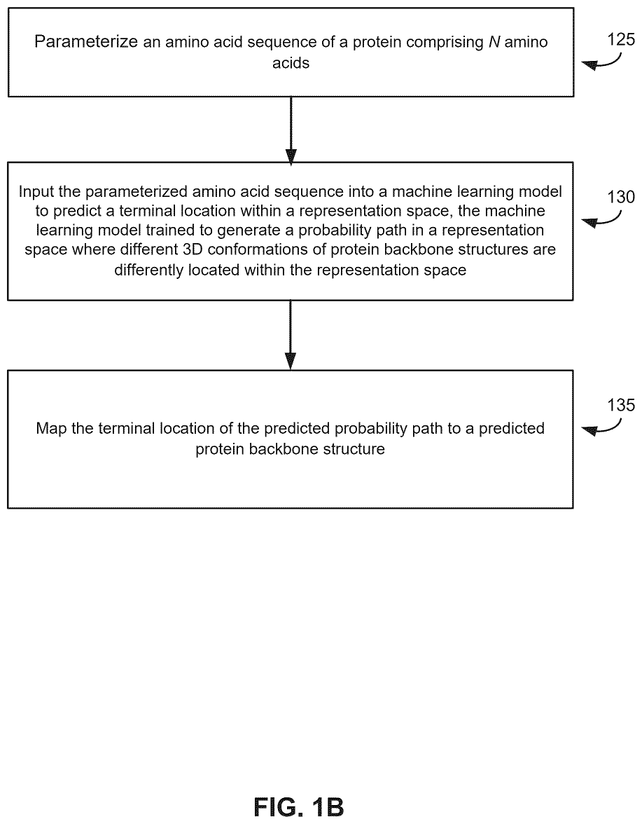

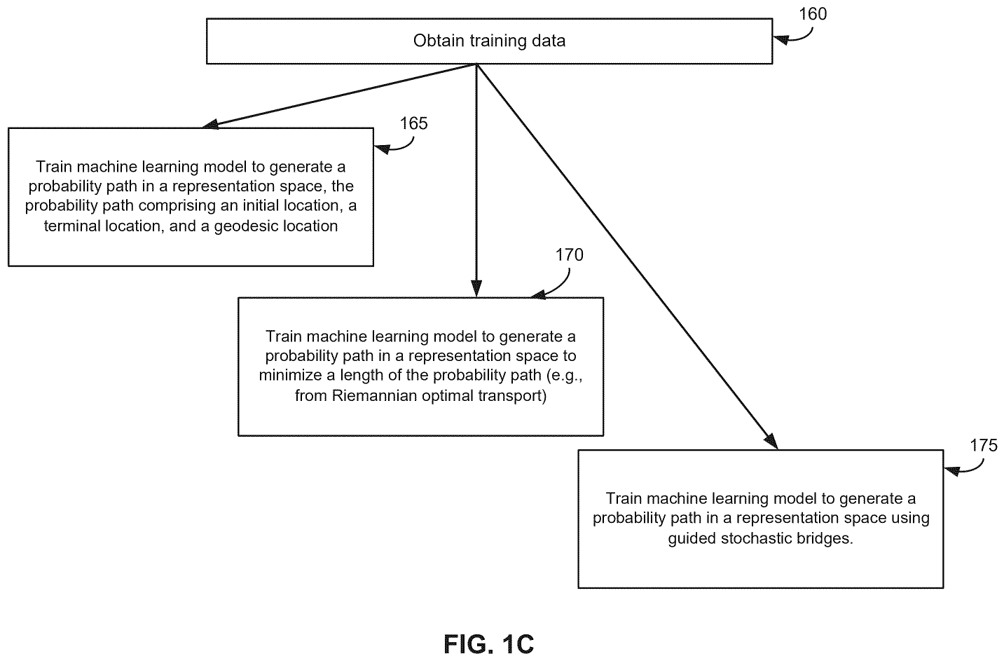

Reference is made to FIG. 1, which depicts an example system overview 100, in accordance with an embodiment. FIG. 1 introduces the protein backbone system 110 and one or more third party entities (e.g., 120A and/or 120B) connected via a network 150.

Generally, the protein backbone system 110 performs methods for predicting protein backbone structures. In various embodiments, the protein backbone system 110 deploys machine learning models e.g., generative predictive models, to predict protein backbone structures. In various embodiments, such machine learning models are trained to generate a probability path in a representation space where different 3D conformations of protein backbone structures are differently located within the representation space. Then, using the terminal location of the predicted probability path, the protein backbone system 110 can predict corresponding protein backbone structures. In various embodiments, such machine learning models are trained to combine the parametrization of a sequence and a structure of a protein into representations (e.g., sequence and structure representations), fuse the representations into joint representations, and decode the joint representations to predict a backbone structure.

Referring to FIG. 1, the protein backbone system 110 may be in communication with one or more third party entities 120. In various embodiments, the third party entity 120 represents a partner entity of the protein backbone system 110 that operates either upstream or downstream of the protein backbone system 110. As one example, the third party entity 120 operates upstream of the protein backbone system 110 and provide information to the protein backbone system 110 to enable the development and/or deployment of machine learning models. In this scenario, the protein backbone system 110 may receive protein data, such as amino acid sequences of proteins. The protein backbone system 110 analyzes the protein data using machine learning models to protein backbone structures. As another example, the third party entity 120 operates downstream of the protein backbone system 110. In this scenario, the protein backbone system 110 generates a predicted protein backbone structure and provides information relating to the predicted protein backbone structure to the third party entity 120. The third party entity 120 can subsequently use the provided information for their own purposes. For example, the third party entity 120 may be a therapeutic developer. Therefore, the therapeutic developer can use the provided information in its investigation, selection, or development of candidate therapeutics.

### Network

This disclosure contemplates any suitable network 150 that enables connection between the protein backbone system 110 and third party entities 120. The network 150 may comprise any combination of local area and/or wide area networks, using both wired and/or wireless communication systems. In one embodiment, the network 150 uses standard communications technologies and/or protocols. For example, the network 150 includes communication links using technologies such as Ethernet, 802.11, worldwide interoperability for microwave access (WiMAX), 3G, 4G, code division multiple access (CDMA), digital subscriber line (DSL), etc. Examples of networking protocols used for communicating via the network 150 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 150 may be represented using any suitable format, such as hypertext markup language (HTML) or extensible markup language (XML). In some embodiments, all or some of the communication links of the network 150 may be encrypted using any suitable technique or techniques.

### Exemplary Methods

### Flow Process for Deploying a Machine Learning Model to Predict a Protein Backbone Structure

Reference is made to FIG. 1B, which depicts a flow diagram for deploying a machine learning model to predict a protein backbone structure, in accordance with an embodiment.

Step 125 involves parameterizing an amino acid sequence of a protein comprising N amino acids.

Step 130 involves inputting the parameterized amino acid sequence into a machine learning model to predict a terminal location within a representation space, the machine learning model trained to generate a probability path in a representation space where different 3D conformations of protein backbone structures are differently located within the representation space.

Step 135 involves mapping the terminal location of the predicted probability to a predicted protein backbone structure.

### Flow Process for Training a Machine Learning Model

Reference is made to FIG. 1C, which depicts a flow diagram for training machine learning models, in accordance with an embodiment. In particular, FIG. 1C shows an example flow diagram for training three different machine learning models.

Step 160 involves obtaining training data.

Step 165 involves training a machine learning model to generate a probability path in a representation space, the probability path comprising an initial location, a terminal location, and a geodesic location. As further described herein, such a machine learning model is referred to as the “FOLDFLOW-BASE” model.

Step 170 involves training a machine learning model to generate a probability path in a representation space to minimize a length of the probability path (e.g., from Riemannian optimal transport). As further described herein, such a machine learning model is referred to as the “FOLDFLOW-OT” model.

Step 175 involves training a machine learning model to generate a probability path in a representation space using guided stochastic bridges. As further described herein, such a machine learning model is referred to as the “FOLDFLOW-SFM” model.

### Additional Flow Diagram for Deploying a Generative Predictive Model to Predict a Protein Backbone Structure

Reference is now made to FIG. 2A, which depicts a block diagram including a generative predictive model for predicting a protein backbone structure, in accordance with an embodiment. In this embodiment, the generative predictive model includes an encoder (labeled as encoder module 215), a fusion trunk (labeled as fusion module 230), and a decoder (labeled as decoder module 245). Generally, the generative predictive model analyzes a sequence and a structure of a protein and generates a prediction 250 e.g., a prediction of a protein backbone structure.

FIG. 2A begins with a structure 205 and a sequence 210. The structure 205 and the sequence 210 may be from the same protein. In various embodiments, the structure 205 and the sequence 210 are each from the same portion of a protein, such as a protein. Thus, the structure 205 represents the 3D conformation of the sequence 210 of the protein.

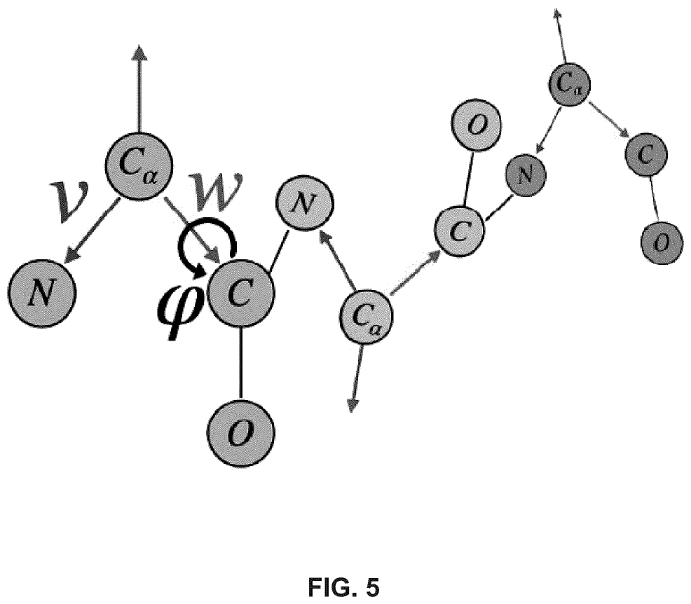

In various embodiments, the structure 205 is a parameterized representation of the structure of a protein and the sequence 210 is a parameterized representation of the sequence of the protein. A parameterized representation refers to an expression of the structure or sequence of the protein using parameters. As an example, the a structure 205 may be parameterized as follows: for a protein of length N this results in N frames that were SE(3)-equivariant. Each frame maps a rigid transformation starting from idealized coordinates of four heavy atoms N*, C_{∞}, C*, O*E [symbol] with C*_{∞}=(0,0,0) being centered at the origin, and is a measurement of experimental bond angles and lengths. Thus, residue i ∈[N] is represented as an action of xⁱ=(rⁱ,sⁱ)∈SE(3) applied to the idealized frame [N, C_{α}, C, O]ⁱ=xⁱ [N*, C*, C*, O*]. To construct the backbone oxygen atom O, rotate about the axis given by the bond between C_{α} and C using an additional rotation angle φ. Finally, denote the full 3D coordinates of all heavy atoms as A∈ [symbol] . An illustration for this backbone parametrization is provided in FIG. 5 with rotations being parametrized as r=v×w.

As shown in FIG. 2A, the structure 205 is inputted into a first portion of an encoder module 215, shown as the “structure encoder”. Additionally, the sequence 210 is inputted into a second portion of the encoder module 215, shown as the “sequence encoder”. The structure encoder and the sequence encoder generate representations of the structure 205 and sequence 210, respectively.

Referring first to the structure encoder, it may include a machine learning model. In various embodiments, the structure encoder can include a neural network including one or more layers of nodes. In various embodiments, the structure encoder includes a transformer architecture. In particular embodiments, the structure encoder is structured with an invariant point attention (IPA) transformer architecture. In particular, the IPA transformer architecture is SE(3)-equivariant. In such embodiments, the IPA architecture is highly flexible and can both consume and produce a structure—i.e., N rigid frames—and also output single and pair representations of the input structure.

As shown in FIG. 2A, the structure encoder outputs three representations e.g., representations in latent space. The three representations include a structure rigid representation 220A, a structure single representation 220B, and a structure pair representation 220C. Generally, a single representation refers to a representation of the tokens in the protein. In various embodiments, a token can refer to an individual amino acid. In various embodiments, a token can refer to an atom. A pair representation represents the relationships (e.g. distance, potential interactions) between all pairs of amino acids/atoms in the protein. The structure rigid representation can refer to a structure of N rigid frames (e.g., a special Euclidean group SE(3) representing a group of rigid body motions or transformations in three-dimensional space).

Referring to the sequence encoder, it may include a machine learning model. In various embodiments, the sequence encoder can include a neural network including one or more layers of nodes. In various embodiments, the sequence encoder comprises a large protein language model, an example of which is the evolutionary scale modeling (ESM) protein language model. Large protein language models have a strong inductive bias on atomic-level predictions of protein structures while exhibiting strong generalization properties beyond any known experimental structures. As shown in FIG. 2A, the sequence encoder generates a sequence single representation 225A and a sequence pair representation 225B. Here, the sequence single representation 225A and a sequence pair representation 225B correspond to the structure single representation 220B and the structure pair representation 220C, respectively. In such embodiments, the output space of each modality prescribes a natural fusion of representations into a joint single and pair latent space for a given input protein.

As shown in FIG. 2A, the representations (e.g., the structure single representation 220B, the structure pair representation 220C, the sequence single representation 225A, and the sequence pair representation 225B are provided as inputs into the fusion module 230. The fusion module 230 may include two portions including a single joint fusion portion and a pair joint fusion portion. Thus, the single joint fusion portion performs a fusion of the single representations (e.g., structure single representation 220B and the sequence single representation 225A). The pair joint fusion portion performs a fusion of the pair representations (e.g., structure pair representation 220C and the sequence pair representation 225B).

In various embodiments, each of the single joint fusion portion and the pair joint fusion portion performs a fusion using a project and concatenate operation. In various embodiments, the project and concatenate operation can be implemented using a neural network, such as a multi-layer perceptron (MLP). In various embodiments, each of the single joint fusion portion and the pair joint fusion portion can further implement Layer Normalization (LayerNorm) to accommodate differently-scaled inputs. In various embodiments, each of the single joint fusion portion and the pair joint fusion portion can further implement one or more folding blocks, which refines the single and pair representations via triangular self-attention updates. The output of the single joint fusion portion is a joint single representation 240A and the output of the pair joint fusion portion is a joint pair representation 240B.

In various embodiments, the generative predictive model includes one or more skip connections between the encoder module 215 and the decoder module 245. For example, as shown in FIG. 2A, each of the structure rigid representation 220A, the structure single representation 220B, and the structure pair representation 220C can be combined and/or inputted into the decoder without having been analyzed by the fusion module 230. For example, the structure rigid representation 220A can be directly inputted into the decoder. As another example, the structure single representation 220B can be combined with the joint single representation 240A and inputted into the decoder. As another example, the structure pair representation 220C can be combined with the joint pair representation 240B and inputted into the decoder.

The decoder takes as input the single representations (e.g., joint single representation 240A combined with structure single representation 220B), the pair representations (e.g., the joint pair representation 240B combined with the structure pair representation 220C), and the structure rigid representation 220A. In various embodiments, the decoder may include a machine learning model. In various embodiments, the decoder can includes a neural network including one or more layers of nodes. In various embodiments, the decoder includes a transformer architecture. In particular embodiments, the decoder is structured with an invariant point attention (IPA) transformer architecture. The decoder outputs a prediction 250, such as a prediction useful for modeling the protein backbone structure.

In particular embodiments, the prediction 250 comprise SE(3)^{N} vector fields. In such embodiments, the prediction 250 is useful for modeling the protein backbone structure. Specifically, SE(3) vector fields are leveraged for transforming a simple source distribution into the protein backbone distribution. Beginning with a random structure, methods involve iteratively refining the structure by applying the vector field, which encodes translational and rotational adjustments in SE(3) space. Using an Euler integration scheme, these updates progressively modify the positions and orientations of atoms, steering the structure toward the target distribution. The iterative process continues until convergence, resulting in a realistic protein backbone that aligns with the learned distribution.

Reference is now made to FIG. 2B, which depicts a flow diagram for implementing a generative predictive model, in accordance with the embodiment shown in FIG. 2A.

Step 260 involves inputting a sequence and a structure of a protein into an encoder to generate a plurality of structure representations and a plurality of sequence representations.

Step 270 involves fusing one or more structure representations and one or more sequence representations to generate at least a joint single representation and a joint pair representation.

Step 280 involves inputting the joint single representation and the joint pair representation into a decoder to generate a prediction useful for modeling the protein backbone structure.

Step 290 may be an optional step that is performed in certain embodiments. Step 290 involves generating or having generated a protein that includes the predicted protein backbone structure. For example, methods can involve synthesizing the protein with the predicted backbone structure using known protein expression/synthesis methods.

### Exemplary Machine Learning Models

Described herein are methods that include deploying machine learning models, such as generative predictive models, for predicting protein backbone structures. In various embodiments, a machine learning model is trained to generate probability paths in a representation space where different 3D conformations of protein backbone structures are differently located within the representation space. In various embodiments, a machine learning model encodes both a sequence and a structure of a protein to generate representations in latent space. The machine learning model fuses the representations in the latent space and generates joint representations which are further decoded (e.g., using a decoder) to generate a prediction of a backbone structure. In various embodiments, the machine learning model is any one of a regression model (e.g., linear regression, logistic regression, or polynomial regression), decision tree, random forest, support vector machine, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks). In particular embodiments, the machine learning model is a generative predictive model including one or more of an encoder, a multi-modal fusion trunk, and a decoder, as is shown in the embodiment in FIG. 2A.

The machine learning model can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the machine learning model is trained using supervised learning algorithms, unsupervised learning algorithms, semi-supervised learning algorithms (e.g., partial supervision), weak supervision, transfer, multi-task learning, or any combination thereof.

In various embodiments, the machine learning model has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, and coefficients in a regression model. The model parameters of the machine learning model are trained (e.g., adjusted) using the training data to improve the predictive power of the machine learning model.

### Non-Transitory Computer Readable Medium

Also provided herein is a computer readable medium comprising computer executable instructions configured to implement any of the methods described herein. In various embodiments, the computer readable medium is a non-transitory computer readable medium. In some embodiments, the computer readable medium is a part of a computer system (e.g., a memory of a computer system). The computer readable medium can comprise computer executable instructions for performing methods described herein.

### Computing Device

The methods described herein are, in some embodiments, performed on a computing device. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

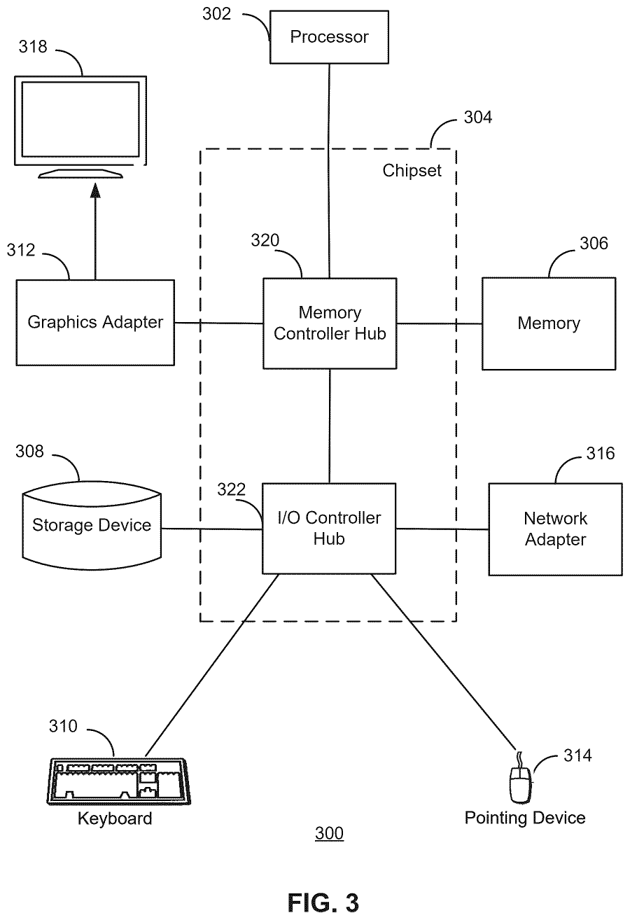

FIG. 3 illustrates an example computing device 300 for implementing system and methods described in FIGS. 1, 2A, and 2B. In some embodiments, the computing device 300 includes at least one processor 302 coupled to a chipset 304. The chipset 304 includes a memory controller hub 320 and an input/output (I/O) controller hub 322. A memory 306 and a graphics adapter 312 are coupled to the memory controller hub 320, and a display 318 is coupled to the graphics adapter 312. A storage device 308, an input interface 314, and network adapter 316 are coupled to the I/O controller hub 322. Other embodiments of the computing device 300 have different architectures.

The storage device 308 is a non-transitory computer-readable storage medium such as a hard drive, compact disk read-only memory (CD-ROM), DVD, or a solid-state memory device. The memory 306 holds instructions and data used by the processor 302. The input interface 314 is a touch-screen interface, a mouse, track ball, or other type of input interface, a keyboard, or some combination thereof, and is used to input data into the computing device 300. In some embodiments, the computing device 300 may be configured to receive input (e.g., commands) from the input interface 314 via gestures from the user. The graphics adapter 312 displays images and other information on the display 318. For example, the display 318 can show an indication of a treatment, such as a treatment validated by applying the cellular disease model. As another example, the display 318 can show an indication of a common chemical structure group likely contributes toward an outcome (e.g., favorable outcome or adverse outcome). As another example, the display 318 can show a candidate patient population that, through implementation of the cellular disease model, has been predicted to respond favorably to an intervention. The network adapter 316 couples the computing device 300 to one or more computer networks.

The computing device 300 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 308, loaded into the memory 306, and executed by the processor 302.

The types of computing devices 300 can vary from the embodiments described herein. For example, the computing device 300 can lack some of the components described above, such as graphics adapters 312, input interface 314, and displays 318. In some embodiments, a computing device 300 can include a processor 302 for executing instructions stored on a memory 306.

The methods described herein can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of displaying any of the datasets and execution and results of a cellular disease model of this invention. Such data can be used for a variety of purposes, such as patient monitoring, treatment considerations, and the like. Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

### Additional Embodiments

Disclosed herein is a method for modeling a protein backbone structure, the method comprising: parameterizing an amino acid sequence of a protein comprising N amino acids; inputting the parameterized amino acid sequence into a machine learning model to predict a terminal location within a representation space, the machine learning model trained to generate a probability path in a representation space where different 3D conformations of protein backbone structures are differently located within the representation space; and mapping the terminal location of the predicted probability path to a predicted protein backbone structure.

In various embodiments, the representation space is a SE(3)^{N} representation space which represents the N amino acids of the amino acid sequence while adhering to symmetries of orientation-preserving rigid transformations. In various embodiments, the representation space is a SO(3) representation space decomposed from a SE(3)^{N} representation space. In various embodiments, the machine learning model maps a first invariant source distribution to a second invariant target distribution over the representation space. In various embodiments, parameterizing the amino acid sequence of the protein comprising N amino acids comprises associating a frame with each of one or more amino acids of the amino acid sequence, wherein each frame maps a rigid transformation of a plurality of atoms of a corresponding amino acid. In various embodiments, the machine learning model is trained to generate a probability path in a representation space, the probability path comprising an initial location, a terminal location, and a geodesic location. In various embodiments, the machine learning model is trained using a loss function of Equation (3).

In various embodiments, the machine learning model is trained to generate a probability path in a representation space to minimize a length of the probability path. In various embodiments, the probability path is generated from Riemannian optimal transport. In various embodiments, the probability path is generated by determining a Monge map. In various embodiments, the machine learning model is trained using a loss function of Equation (5). In various embodiments, the machine learning model is trained to generate a probability path in a representation space using guided stochastic bridges. In various embodiments, a guided stochastic bridge represents guided Brownian motion from an initial location of the probability path to a terminal location of the probability path.

In various embodiments, the machine learning model is trained using a loss function of Equation (9). In various embodiments, the machine learning model is trained using an auxiliary loss value. In various embodiments, the auxiliary loss value is a loss on pairwise atomic distances. In various embodiments, the N amino acids comprise at least 50, at least 100, at least 150, at least 200, at least 200, or at least 300 amino acids.

In various embodiments, the method generates improved protein backbone structures as measured by one or more of designability, diversity, or novelty in comparison to FrameDiff, Genie, or RFDiffusion. In various embodiments, the machine learning model is a generative machine learning model. In various embodiments, the machine learning model reduces training time by at least 2-fold in comparison to FrameDiff, Genie, or RFDiffusion. In various embodiments, the method does not involve computing a Gaussian distribution score.

Additionally disclosed herein is a non-transitory computer readable medium comprising instructions that, when executed by a processor, cause the processor to perform any of the methods disclosed herein. Additionally disclosed herein is a system comprising: a processor; and a non-transitory computer readable medium comprising instructions that, when executed by a processor, cause the processor to perform any of the methods disclosed herein.

### Enumerated Embodiments

Embodiments provided herein also include, but are not limited to:

1. A method for modeling a protein backbone structure, the method comprising:
inputting a sequence and a structure of a protein into an encoder to generate a plurality of structure representations and a plurality of sequence representations;
fusing one or more structure representations and one or more sequence representations to generate at least a joint single representation and a joint pair representation; and
inputting the joint single representation and the joint pair representation into a decoder to generate a prediction useful for modeling the protein backbone structure.

2. The method of embodiment 1, wherein inputting the sequence and the structure of the protein into the encoder comprises parameterizing the sequence and a backbone of the protein.

3. The method of claim 1 or 2, wherein inputting the sequence and the structure of the protein into the encoder further generates a rigid representation.

4. The method of embodiment 3, wherein the rigid representation includes a special Euclidean group SE(3) representing a group of rigid body motions or transformations in three-dimensional space.

5. The method of any one of embodiments 1-4, wherein the encoder comprises a structure encoder and a sequence encoder.

6. The method of embodiment 5, wherein the sequence encoder comprises a protein language model.

7. The method of embodiment 5, wherein the structure encoder comprises an invariant point attention (IPA) transformer architecture.

8. The method of any one of embodiments 1-7, wherein the fusing of one or more structure representations and one or more sequence representations is performed using a multi-modal fusion trunk which combines multi-modal representations of encoded structure representations and sequence representations.

9. The method of embodiment 8, wherein the multi-modal fusion trunk performs adaptive fusion of the one or more structure representations and one or more sequence representations, wherein the adaptive fusion comprises dynamically adjusting the fusion based on the complexity or variability of the sequence and/or the structure of the protein.

10. The method of embodiment 8, wherein the decoder consumes the joint single representation and the joint pair representation from the multi-modal fusion trunk and outputs the prediction comprising a generated structure.

11. The method of any one of embodiments 1-10, wherein one or more skip connections are present between the encoder and the decoder.

12. The method of any one of embodiments 1-11, wherein the encoder and the decoder are structured within a generative prediction model.

13. The method of embodiment 12, wherein the generative prediction model further comprises a multi-modal fusion trunk.

14. The method of embodiment 12 or 13, wherein the generative prediction model considers inductive bias of amino acid sequences.

15. The method of any one of embodiments 12-14, wherein the generative prediction model uses flow matching.

16. The method of embodiment 15, wherein the generative prediction model uses SE(3) Equivariant flow matching.

17. The method of embodiment 16, wherein the SE(3) equivariant flow matching comprises probability paths on SO(3) and/or matching vector fields on SO(3).

18. The method of any one of embodiments 12-17, wherein the generative predictive model is SE(3)^{N}-invariant.

19. The method of any one of embodiments 12-17, wherein the generative predictive model uses an SE(3)^{N}-invariant density using a flow-matching objective.

20. The method of any one of embodiments 12-17, wherein the generative predictive model is translation invariant.

21. The method of any one of embodiments 15-20, wherein performing flow matching comprises building flows on a group of rotations SO(3) and translation R3.

22. The method of embodiment 21, wherein building flows on the group of rotations SO(3) and translation R3 for each of N residues independently as SE(3) is a product manifold comprising N copies of SE(3).

23. The method of any one of embodiments 12-22, wherein the generative predictive model parametrizes protein backbones x₀˜ρ₀ as N rigid frames.

24. The method of any one of embodiments 12-23, wherein the generative predictive model is a multi-modal generative predictive model.

25. The method of any one of embodiments 12-23, wherein the generative predictive model is trained using one or more loss functions.

26. The method of embodiment 25, wherein the one or more loss functions comprise hierarchical loss functions that balance global structural fidelity with local sequence accuracy during training.

27. The method of embodiment 25, wherein the one or more loss functions comprise a loss function that decomposes into per residue rotation and translation.

28. The method of embodiment 27, wherein the loss function is

$$L = {E}_{t ∼ U ( 0 , 1 ) , {ρ}_{t} ( {x}_{0} , {x}_{1} , \overline{a} )} [ {‖ {v}_{θ} ( t , {r}_{t} , \overline{a} ) - {log}_{{r}_{t}} ( {r}_{0} ) / t ‖}_{SO ( 3 )}^{2} + {‖ {v}_{θ} ( t , {s}_{t} , \overline{a} ) - \frac{{s}_{t} - {s}_{0}}{t} ‖}_{2}^{2}$$

29. The method of any one of embodiments 12-28, wherein the generative predictive model is trained by curating and/or filtering datasets of protein structure using one or more of steps of:
a) filtering low-confidence structures using per-residue local confidence metrics to filter out low-confidence structures;
b) masking low-confidence residues; and
c) filter high-confidence, low-quality structures by learning a structure prediction model trained on structural features predictive of protein quality.

30. The method of any one of embodiments 12-28, wherein the generative predictive model is trained by alternating between both folding and generation tasks.

31. The method of any one of embodiments 12-28, wherein the generative predictive model is trained by keeping a pre-trained language model fixed while training the encoder, multi-modal fusion trunk, and decoder.

32. The method of any one of embodiments 12-31, wherein the generative predictive model is trained using partially masked sequences or structures, thereby enabling one or more of folding tasks, inverse folding tasks, conditional design of protein complexes, and sequence-structure co-design.

33. The method of any one of embodiments 12-31, wherein the generative predictive model is trained using transfer learning techniques.

34. The method of any one of embodiments 12-31, wherein the generative predictive model further incorporates environmental or experimental factors during training.

35. The method of embodiment 34, wherein environmental or experimental factors comprise pH, temperature, or binding partners.

36. The method of any one of embodiments 12-31, wherein the generative predictive model is trained using cross-modal data augmentation techniques to enhance the robustness of the generative predictive model to incomplete or noisy inputs in either sequence or structure domains.

37. The method of any one of embodiments 12-31, wherein the generative predictive model is trained using a reinforcement fine-tuning approach that aligns generation outputs with desired biological properties.

38. The method of embodiment 37, wherein desired biological properties comprise binding affinity or stability.

39. The method of any one of embodiments 12-31, wherein the generative predictive model further incorporates protein evolutionary information, thereby improving biological relevance of the prediction useful for modeling the protein backbone structure.

40. The method of any one of embodiments 12-31, wherein the generative predictive model leverages ensemble methods during training and inference to improve the diversity and reliability of the prediction useful for modeling the protein backbone structure.

41. The method of any one of embodiments 12-31, wherein the generative predictive model predicts and/or optimizes equilibrium conformations of proteins using latent space perturbations.

42. The method of any one of embodiments 1-41, further comprising using the modeled protein backbone structure for one or more of:
a) unconditional protein backbone generation;
b) increasing secondary structure diversity;
c) protein sequence folding;
d) protein structure motif scaffolding;
e) protein equilibrium conformation sampling;
f) capturing different modes of the equilibrium conformation;
g) partial structure generation by conditioning on a masked sequence;
h) de novo drug design; and
i) engineering a structure that binds a desired target protein structure and sequence pair.

43. The method of any one of embodiments 1-42, wherein the one or more structure representations and one or more sequence representations represent different representations of protein structures at different resolutions.

44. The method of any one of embodiments 1-43, further providing a visualization of sequence motifs or structural elements in the fused one or more structure representations and one or more sequence representations, thereby enabling interpretability in the prediction useful for modeling the protein backbone structure.

45. The method of any one of embodiments 1-44, wherein the inputted sequence of the protein includes one or more masked amino acids.

46. The method of embodiment 45, wherein the generative predictive model performs in-painting tasks for resolving the one or more masked amino acids.

47. The method of any one of embodiments 1-44, wherein the inputted sequence of the protein includes one or more functional motifs or catalytic sites.

48. The method of embodiment 47, wherein the generative predictive model generates the prediction useful for modeling the protein backbone structure while optimizing for the one or more functional motifs or catalytic sites.

49. A method for modeling a multi-protein complex, the method comprising:
inputting a sequence and a structure of a protein into an encoder to generate a plurality of structure representations and a plurality of sequence representations;
fusing one or more structure representations and one or more sequence representations to generate at least a joint single representation and a joint pair representation; and
inputting the joint single representation and the joint pair representation into a decoder to generate a prediction.

50. The method of embodiment 49, wherein the encoder further receives as input inter-protein interactions between the protein and a corresponding protein in the multi-protein complex, and the decoder generates the prediction useful for determining the inter-protein interaction.

51. The method of embodiment 50, wherein the decoder simultaneously generates predictions useful for determining the inter-protein interaction and one or more protein backbone sequences.

52. A circuit for a generative predictive model for modeling a protein backbone structure, the generative predictive model comprising:
an encoder comprising a plurality of neurons for analyzing a sequence and a structure of a protein to generate a plurality of structure representations and a plurality of sequence representations;
a multi-modal fusion trunk communicatively coupled to the encoder, wherein the multi-modal fusion trunk fuses one or more structure representations and one or more sequence representations to generate at least a joint single representation and a joint pair representation; and
a decoder comprising a second plurality of neurons communicatively coupled to the multi-modal fusion trunk for analyzing the joint single representation and the joint pair representation to generate a prediction useful for modeling the protein backbone structure.

53. The circuit of embodiment 52, wherein the first plurality of neurons and/or the second plurality of neurons comprise one or more registers and one or more microprocessors.

54. The circuit of embodiment 52 or 53, wherein the circuit further comprises a plurality of synaptic circuits including one or more memories for storing model weights, wherein the model weights are learned via training of the generative predictive model and are included in any of the encoder, the multi-modal fusion trunk, and the decoder.

### EXAMPLES

### Example 1: Example Machine Learning Models (e.g., FOLDFLOW Models) Outperform Conventional Methods in Generating Protein Backbone Structures

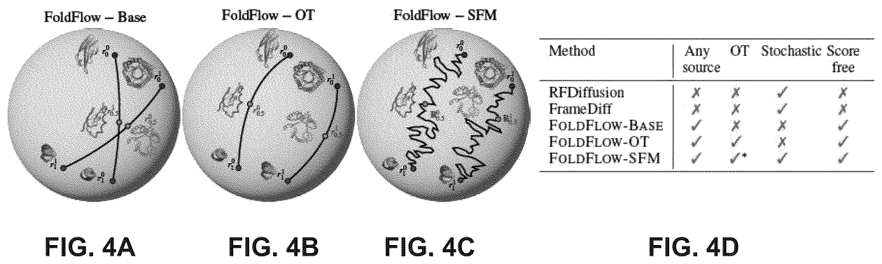

The computational design of novel protein structures has the potential to impact numerous scientific disciplines greatly. Toward this goal, introduced herein is FOLDFLOW, a series of novel generative models of increasing modeling power based on the flow-matching paradigm over 3D rigid motions—i.e. the group SE(3)—enabling accurate modeling of protein backbones. A first FOLDFLOW model is termed “FOLDFLOW-BASE,” a simulation-free approach to learning deterministic continuous-time dynamics and matching invariant target distributions on SE(3). A second FOLDFLOW model is termed “FOLDFLOW-OT” which undergoes accelerated training by incorporating Riemannian optimal transport, leading to the construction of both more simple and stable flows. Finally, a third FOLDFLOW model is termed “FOLDFLOWSFM,” which couples both Riemannian OT and simulation-free training to learn stochastic continuous-time dynamics over SE(3). The family of FOLDFLOW generative models offers several key advantages over previous approaches to the generative modeling of proteins: they are more stable and faster to train than diffusion-based approaches, and the FOLDFLOW models enjoy the ability to map any invariant source distribution to any invariant target distribution over SE(3). Empirically, the FOLDFLOW models were validated on protein backbone generation of up to 300 amino acids leading to high-quality designable, diverse, and novel samples. FIGS. 4A-4C show the conditional probability paths learned by the various FOLDFLOW models. Where FOLDFLOW-BASE paths may cross, FOLDFLOW-OT conditional paths do not cross reducing the variance in the objective stabilizing training as studied in Pooladian et al. (2023); Tong et al. (2023b). FOLDFLOW-SFM adds in stochasticity which improves novelty in the protein generation task. FIG. 4D contains a table summarizing the differences between methods. Showing whether or not they can map from a general source distribution, can perform optimal transport under some conditions, are stochastic or deterministic, and do not require calculation of the score. There is a * for FOLDFLOW-SFM performing OT, as it only achieves OT when noise goes to zero and it recovers FOLDFLOW-OT However, this bias may still be helpful in reducing the variance of the objective function even if OT is not achieved.

### Background and Preliminaries

### Riemannian Manifolds.

Roughly speaking, an n-dimensional manifold is a topological space locally equivalent (homeomorphic) to [symbol] . This implies that one has the notion of ‘neighbourhood’ but not of ‘distance’ or ‘angle’ on M. The manifold is said to be smooth if it additionally has a C^{∞} differential structure. At every point x∈M, one can attach a tangent space Tₓ. The collection (disjoint union) of tangent spaces forms the tangent bundle. A Riemannian manifold (M, g) is additionally equipped with an inner product (Riemannian metric) gx: TₓM xTₓM→ [symbol] on the tangent space TₓM at each x∈M. The Riemannian metric g allows to define key geometric quantities on M such as distances, volumes, angles, and length minimizing curves (geodesics). Functions defined on M and the tangent bundle, referred to as scalar- and vector fields, respectively, are considered. The Riemannian gradient is an operator ∇_{g}: C^{∞}(M)→X(M) between the respective functional spaces. Given a smooth scalar field f∈C^{∞}(M), its gradient ∇_{g}f∈X(M) is the local direction of its steepest change.

### Lie Groups

A Lie group is a group that is also a differentiable manifold, in which the group operations o: G×G→G of multiplication and inversion are smooth maps. It has a left action Lₕ: G→G defined by x→h o x that is a topological isomorphism and whom the derivative is also an isomorphism between the tangent spaces on G. Since a group has an identity element, its tangent space is of special interest and is known as the Lie algebra. The Lie algebra is a vector space with an associated bilinear operation called the Lie bracket that is anticommutative and satisfies the Jacobi identity. Lie algebras elements can be mapped to the Lie group via the exponential map map exp: G→G which has an inverse called the logarithmic map log: G→G. For matrix Lie groups where the group action is the matrix multiplication, the exp and log maps correspond to the matrix exponential and matrix logarithm. The orientation-preserving rigid motions form the matrix Lie group SE(3)≈SO(3) [symbol] ( [symbol] , +), a semidirect product of rotations and translations.

### Flow Matching on Riemannian Manifolds

Analogous to Euclidean spaces, probability densities can be defined on Riemannian manifolds as continuous non-negative functions ρ: M→ [symbol] , that integrate to

∫_{M}ρ(x)dx=1.

Probability paths on Riemannian manifolds. Let P(M) be the space of probability distributions on M. A probability path ρₜ: [0,1]→P(M) is an interpolation in probability space between two distributions ρ₀, ρ₁ ∈P(M) indexed by a continuous parameter t. A one-parameter diffeomorphism ψₜ: M→M is known as a flow on and is defined as the solution of the following ordinary differential equation (ODE):

$$\frac{d}{dt} {ψ}_{t} ( x ) = {u}_{t} ( {ψ}_{t} ( x ) ) ,$$

with initial conditions ψ₀(x)=x, for u: [0, 1]×M→M a time-dependent smooth vector field. The flow ψₜ generates ρₜ if it pushes forward ρ₀ to ρ₁ by following the time-dependent vector field uₜ—i.e.

$${ρ}_{t} = [ {ψ}_{t} ] ( {ρ}_{0} ) .$$

As ψₜ is a diffeomorphism, ρₜ verifies the famous continuity equation and the density can be calculated using the instantaneous change of variables formula for Riemannian manifolds (Mathieu & Nickel, 2020).

Riemannian flow matching. Given a probability path ρₜ that connects ρ₀ to ρ₁, and its associated flow ψₜ, learn a CNF by directly regressing the vector field uₜ with a parametric one νθ∈X(M). This technique is termed flow matching (Lipman et al., 2022, FM) and leads to a simulation-free training objective as long as ρₜ satisfies the boundary conditions ρ₀=ρ_{data} and ρ₁=ρₚᵣᵢₒᵣ. Unfortunately, the vanilla flow matching objective is intractable given no access to the closed-form of uₜ that generates ρₜ. Instead, opt to regress ν_{θ} against a be recovered by marginalizing of conditional vector fields:

$${u}_{t} ( x ) := {\int }_{M} {u}_{t} ( x \vert z ) \frac{( {ρ}_{t} ( {x}_{t} \vert z ) q ( z ) )}{{ρ}_{t} ( x )} dz .$$

The Riemannian CFM objective (Chen & Lipman, 2023) is then,

$${L}_{rcfm} ( θ ) = {E}_{t , q ( z ) , {ρ}_{t} ( {x}_{t} \vert z )} {‖ {v}_{θ} ( t , {x}_{t} ) - {u}_{t} ( {x}_{t} \vert z ) ‖}_{g}^{2} , t ∼ u ( 0 , 1 ) \qquad ( 1 )$$

As FM and CFM objectives have the same gradients (Tong et al., 2023b), at inference, generate by sampling from ρ₁, and using ν_{θ} to propagate the ODE backward in time.

### Protein Backbone Parameterization

The protein backbone parameterization follows the seminal work of AlphaFold2 (Jumper et al., 2021, AF2) in that a frame was associated with each residue in the amino acid sequence. For a protein of length N this results in N frames that were SE(3)-equivariant. Each frame maps a rigid transformation starting from idealized coordinates of four heavy atoms N*, C_{∞}, C*, O*∈ [symbol] with C*_{∞}=(0,0,0) being centered at the origin, and is a measurement of experimental bond angles and lengths. Thus, residue i∈[N] is represented as an action of xⁱ=(rⁱ, sⁱ)∈SE(3) applied to the idealized frame [N, C_{α}, C, O]ⁱ=xⁱ o[N*, C*, C*, O*]. To construct the backbone oxygen atom O, rotate about the axis given by the bond between C_{α}and C using an additional rotation angle φ. Finally, denote the full 3D coordinates of all heavy atoms as A∈ [symbol] . An illustration for this backbone parametrization is provided in FIG. 5 with rotations being parametrized as r=v×w.

### FOLDFLOW For Condition Flow Matching On SE(3)

FOLDFLOW models were trained to learn an SE(3)^{N} invariant density ρₜ by training a flow using the objective in eq. (1). A SE(3)^{N}-invariant source distribution ρ₁ was pushforward to the empirical distribution of proteins ρ₀ using an equivariant flow. One way to guarantee the existence of a translation-invariant measure is to construct a subspace that is invariant to global translations. This can be achieved by simply subtracting the center of mass of all inputs to the flow (Rudolph et al., 2021; Yim et al., 2023b). Formally, this leads to an invariant measure on

$${SE ( 3 )}_{0}^{N}$$

which is a subgroup of SE(3)^{N}. SE(3)^{N} is a product group and thus the Riemannian metric extends in a natural way to the product space: the exponential and logarithmic maps decompose across each manifold, and the geodesic distance in

$$SE {( 3 )}_{0}^{N}$$

is simply the sum of each individual distance in the product. As such, a flow on SE(3)₀^{N} can be built from separate flows for each residue in the backbone, on SE(3), after centering.

Decomposing SE(3) into SO(3) and [symbol] . As Lie groups are manifolds, they can be equipped with a metric to obtain a Riemannian structure. In the case of SE(3)≈SO(3) ( [symbol] , +) there are multiple possible choices, but a natural one is <x, x′>SE(3)=<r,r′>so(3)+<s, s′ [symbol] . Moreover, the disintegration of measures implies that every SE(3)-invariant measure can be broken down to a SO(3)-invariant measure and a measure proportional to the Lebesgue measure on [symbol] (Pollard, 2002). Thus, independent flows were built on SO(3) and [symbol] . The notations ρₜ, q, and π are used for densities whose support is determined by its context.

### Foldflow-Base

To construct a flow on SO(3) that connects the target distribution ρ₀ to a source distribution ρ₁, first choose a parametrization of the group elements. The most familiar and natural parametrization is by orthogonal matrices with unit determinant. The Lie algebra so (3) contains skew-symmetric matrices r that are tangent vectors at the identity of SO(3). The last component is a choice of Riemannian metric for SO(3). A canonical bi-invariant metric for SO(3) can be derived from the Killing form, and is given by:

$${〈 r , {r}^{′} 〉}_{SO ( 3 )} = \frac{tr ( {rr}^{′ T} )}{2} .$$

SO(3) conditional vector fields and flows. Seek to construct a conditional vector field uₜ (rₜ|z), lying on the tangent space Tᵣ_{ₜ} SO(3), that transports r₀˜ρ₀ to r₁˜ρ₁. Following, Tong et al. (2023b) set the conditioner to z=(r₀, r₁). Next, construct a flow ψ that connects r₀ to r₁. The flow was built using the geodesic between r₀ and r₁. For general M, the geodesic interpolant between two points, indexed by t, has the following form:

$${r}_{t} = {exp}_{r 0} ( t {log}_{r 0} ( {r}_{1} ) ) \qquad ( 2 )$$

For rotation matrices, eq. (2) involves computing the exp and log maps which are both infinite matrix power series. Unfortunately, controlling the approximation error of logᵣ₀ map is computationally expensive as the de facto inverse scaling method for computing matrix logarithms requires estimating and calculating fractional matrix powers (Al-Mohy & Higham, 2012). Instead, use a numerical trick by converting r₁ to its axis-angle representation which gives a vector representation of r₁ ∈so(3) and, by definition, lives at the tangent space at the identity and is equivalent to logₑ (r₁). Next, parallel transport r₁ to the tangent space of r₀ since Lie algebras of all tangent spaces are isomorphic and SO(3) carries a free action which gives the desired end result logᵣ₀ (r₁).

Given rₜ, build constant velocity vector fields by directly leveraging the ODE associated with the conditional flow:

$$\frac{d}{dt} {ψ}_{t} ( r ) = {r}^{\cdot } t$$

(Chen & Lipman, 2023). As a result, computing uₜ (rₜ|z) boils down to computing the point rₜ along the ODE and taking its time derivative. In practice, taking the time-derivative to compute uₜ=r′t amounts to using autograd to compute the gradient during a forward pass. This overhead was overcome without relying on automatic differentiation but instead by exploiting the geometry of the problem. Specifically, calculate the so(3) element corresponding to the relative rotation between r₀ and rₜ, given by

$${r}_{t}^{T} {r}_{0} .$$

Divide by t to get a vector which is an element of so(3) and corresponds to the skew-symmetric matrix representation of the velocity vector pointing towards the target r₁. Finally, parallel-transport the velocity vector to the tangent space rₜ SO(3) using left matrix multiplication by rₜ. These operations can be concisely written as logᵣ_{ₜ} (r₀) and the matrix logarithm is calculated. The closed form expression of the loss to train the SO(3) component of FOLDFLOW-BASE is thus

$${L}_{FOLDFLOW - BASE - SO ( 3 )} ( θ ) = {E}_{t ∼ u ( 0 , 1 ) , q ( {r}_{0 ,} {r}_{1} ) , {ρ}_{t} ( {r}_{t} \vert {r}_{0} , {r}_{1} )} ‖ {v}_{θ} ( t , {r}_{t} ) - {log}_{{r}_{t}} {r}_{0} ) / t {\vert}_{SO ( 3 )}^{2} \qquad ( 3 )$$

In eq. (3) the conditioning distribution q(z)=q(r₀, r₁) is the independent coupling q(r₀, r₁)=ρ₀ρ₁, where ρ₁=U(SO(3)) and is left-invariant w.r.t. to the Haar measure. Also, note that the vector field in eq. (3) is on the tangent space ν_{θ}∈Tᵣ_{ₜ} SO(3) and the norm is induced by the metric on SO(3).

### Foldflow-Ot

The conditional vector field uₜ (rₜ|z) generates the conditional probability path ρₜ (rₜ|z) which deterministically evolves ρ₀ to ρ₁. However, there is no reason to believe the conditional probability path is optimal in the sense that it is a length-minimizing curve, under an appropriate metric, in the space of distributions P(SO(3)). Therefore, this is rectified by constructing conditional probability paths that are not only shorter and straighter, but also more stable from an optimization perspective.

FOLDFLOW-OT is a model that accelerates FOLDFLOW-BASE by constructing conditional probability paths using Riemannian optimal transport. The interpolation measure ρₜ connects ρ₀→ρ₁ and is built from Riemannian OT which solves the Monge optimal transport problem:

$$OT ( {ρ}_{0} , {ρ}_{1} ) = \begin{matrix}inf \\ Ψ : Ψ \neq {ρ}_{0} = {ρ}_{1}\end{matrix} {\int }_{{SE ( 3 )}_{0}^{N}} \frac{1}{2} {c ( x , Ψ ( x ) )}^{2} d {ρ}_{0} ( x ) \qquad ( 4 )$$

Here c is the geodesic cost induced by the metric and Y′ a pushforward map: ρ₀→ρ₁. A related problem, called the OT-Kantorovich formulation, relaxes the Monge problem by looking for a joint probability distribution I minimizing the displacement cost of transporting ρ₀ to ρ₁. The uniqueness of the Monge map over SE(3)^{N} is guaranteed under some assumptions on the measures ρ₀,ρ₁.

Define the McCann interpolants as ρₜ(x)=(expₓ (t∇ϕ(x))) #ρ₀. While it is possible to approximate the Monge map and McCann interpolants using c-concave functions, it imposes practical limitations on the architecture of the flow (Cohen et al., 2021). Instead, use the correspondence between the Monge and Kantorovich problems and rely on the optimal transport plan π. Formally, draw two samples from q(z)=q(x₀, x₁): =π(x₀, x₁) and compute for a given frame ρₜ (rₜ|r₀, r₁)=δ(expᵣ_{₀} (tlogᵣ_{₀}(r₁), where δ is a Dirac. Since the choice of metric for SE(3) factorizes into metrics on SO(3) and [symbol] , independent losses can be used on rotations and translations—similar to FOLDFLOW-BASE—and repeat this over N frames, as long as each geometric quantity in x is coupled properly. Defining \hat{π}(r₀, r₁) as the projection of π(x₀, X₁) on SO(3), the SO(3) loss for a single frame in FOLDFLOW-OT is expressed as:

$${L}_{FOLDFLOW - OT - SO ( 3 )} ( θ ) = {E}_{t ∼ u ( 0 , 1 ) , \overline{π} ( {r}_{0 ,} {r}_{1} ) , {ρ}_{t} ( {r}_{t} \vert {r}_{0} , {r}_{1} )} ‖ {v}_{θ} ( t , {r}_{t} ) - {log}_{{r}_{t}} {r}_{0} ) / t {\vert}_{SO ( 3 )}^{2} \qquad ( 5 )$$

### Foldflow-Sfm

The third model, referred to as FOLDFLOW-SFM, builds on the foundations of both FOLDFLOW-BASE and FOLDFLOW-OT. Departing from the deterministic dynamics of the previous models, build a stochastic flow over SE(3)^{N} by replacing these deterministic bridges with guided stochastic bridges. In Euclidean space, a translation invariant flow was built on

$${( {ℝ}^{3} )}_{0}^{N}$$

by using a (reverse time) brownian bridge as the conditional flow between the points,

$${dS}_{t} = \frac{{S}_{t} - {s}_{0}}{t} dt + γ ( t ) {dW}_{t} , {S}_{1} = {s}_{1} \qquad ( 6 )$$

This flow, also known as Doob's h-transform (Doob, 1984), is easy to sample from in a simulation-free manner and correctly maps between arbitrary samplable marginals in expectation. Specifically, a simulation-free bridge was built by sampling from the conditional probability ρₜ (xₜ|x₀, x₁)=N (xₜ; tx₁+(1−t) x₀, γ²t(1−t) (Shi et al., 2023; Albergo et al., 2023).

Brownian Bridge on SO(3). Using a guided diffusion bridge, model the dynamics between rotations matrices on SO(3) (Jensen et al., 2022; Liu et al., 2022), thereby leading to the following dynamics,

$$d {R}_{t} = \frac{{log}_{{R}_{t}} {r}_{0}}{t} d t + γ ( t ) d {B}_{t} , {R}_{1} = {r}_{1} \qquad ( 7 )$$

for Bₜ the Brownian motion on SO(3). Despite the close resemblance of this SDE to the translation one in eq. (6), the corresponding Brownian bridge does not have a closed-form expression for ρₜ (rₜ|r₀, r₁). Thus, to sample from ρₜ (rₜ|r₀, r₁) correctly, start at r₁ and simulate the bridge backward in time. Given this form of the conditional bridge, make use of a flow-matching loss to optimize a flow between arbitrary source and target distributions. Specifically, define the stochastic flow-matching loss on SO(3) as:

$${{L}_{SFM - SO ( 3 )} ( θ ) = {E}_{t ∼ u ( 0 , 1 ) , \overline{π} ( {r}_{0} , {r}_{1} ) , {ρ}_{t} ( {r}_{t} \vert {\overline{r}}_{0} , {r}_{1} )} ‖ {v}_{θ} ( t , {\overline{r}}_{t} ) - {log}_{{\overline{r}}_{t}} {r}_{0} ) / t \vert}_{SO ( 3 )}^{2} \qquad ( 8 )$$

Here It is a sample from the bridge between r₀ and r₁. When π(x₀, x₁) is a valid coupling between ρ₀ and ρ₁ and thus π̅(r₀, r₁) on SO(3)—this objective is equivalent in expectation to matching directly the (computationally intractable) marginal loss

$${L}_{U S F M} = {E}_{t ∼ u ( 0 , 1 ) , {ρ}_{t} ( {\overline{r}}_{t} )} {‖ {v}_{θ} ( t , {\tilde{r}}_{c} ) - u ( t , {\tilde{r}}_{t} ) ‖}_{SO 3}^{2} .$$

This result allows the learning of a stochastic flow from any source to any target distribution supported on SE(3)^{N}, only involving samples from both distributions. However, it does involve simulation of an SDE to sample from the conditional probability ρₜ (rₜ|r₀, r₁), limiting scalability.

An Efficient Simulation-free Approximation. Sampling from the correct conditional bridge requires simulation and is thus computationally expensive for training. In practice, a simulation-free approximation that closely matches the true conditional probability path on SO(3), ρₜ (\tilde{r}ₜ|r₀, r₁) is used. Specifically, approximate ρₜ with the simulation-free alternative, \hat{ρ}ₜ (\tilde{r}ₜ|r₀, r₁)=IGₛ₀₍₃₎ \tilde{r}ₜ; expᵣ_{₀}(t logᵣ_{₀}(r1)), γ² (t) t(1−t)(9) where IGₛ₀₍₃₎ denotes the isotropic Gaussian distribution on SO(3). This distribution can be seen as an analog of the Gaussian distribution in Rd. It is the heat kernel on SO(3) (Nikolayev & Savyolov, 1900) and it can be seen as the limit of small i.i.d. rotations in 3D (Qiu, 2013). Additionally, it has some of the desirable properties of the normal distribution, such as being closed under convolution (Nikolayev & Savyolov, 1900).

### Modeling Protein Backbones Using FOLDFLOW

To model protein backbones using FOLDFLOW models, parameterize the velocity prediction ν_{θ(t,x}_{ₜ}₎ as a function that consumes a protein Xₜ on the conditional path at time t and predicts the starting point \hat{x}₀. Specifically, the predicted velocity is

$${v}_{θ} ( t , {x}_{t} ) = \frac{{∇}_{g} {d ( {\hat{x}}_{0} , {x}_{t} )}^{2}}{t} ,$$

with \hat{x}₀=W_{σ}(t, xₜ). This choice of parameterization has two principal benefits. (1) It allows the usage of specialized architectures specifically designed for structure prediction, and (2) it allows for auxiliary protein-specific losses to be placed directly on the x\hat{ }0 to improve performance.

Architecture. Following prior work by Anand & Achim (2022); Yim et al. (2023b) the structure module of AF2 was used to model w_{θ}. This begins with a time-dependent node and edge embeddings N_{θ}(t, xₜ) and Ee (t, xₜ), followed by layers of invariant point attention. A small MLP head was usedon top of the node embeddings to predict the torsion angle of the oxygen φ as \hat{φ}=MLP (N_{θ}(t, xₜ)).

Full Loss. The FOLDFLOW models were trained to optimize a flow-matching loss on SO(3) and [symbol] for each residue i ∈[N] in the backbone. These are denoted FOLDFLOW-SO(3) and FOLDFLOW- [symbol] respectively. In addition to the flow-matching losses, auxiliary losses were also included which enforced good predictions at the atomic level in the [symbol] atomic representation A. These included a direct regression on the backbone (bb) positions L_{bb} and a loss on the pairwise atomic distance in a local neighbourhood L_{2D},

$${L}_{a u x} = {E}_{Q} [ {L}_{b b} + {L}_{2 D} ] , {L}_{b b} = \frac{1}{4 N} \sum {‖ {A}_{0} - {\hat{A}}_{0} ‖}^{2} ,$$

$${L}_{2 D} = \frac{{‖ 1 \{ D < 6 \dot{A} \} ( D - \hat{D} ) ‖}^{2}}{\sum {1}_{D < 6 \dot{A}} - N}$$

Here Q (t, X₀, X₁, \tilde{X}ₜ): =U(0, 1)⊗π̅(x₀, x₁)⊗ρₜ(\tilde{X}ₜ|x₀, x₁) is the factorized joint distribution, 1 is the indicator function, A is in Angstroms (Å), D is an N×N×4×4 tensor containing the pairwise distances between the four heavy atoms, i.e. D_{ijab}=∥Aᵢₐ-A_{jb}∥, and D\hat{ } is defined similarly from A\hat{ }. Only apply auxiliary losses for t<0.25, with scaling λₐᵤₓ for a final loss of:

$${L}_{FOLDFLOW} ( θ ) = {L}_{F O L D F L O W - SO ( 3 )} + {L}_{F O L D F LOW -} + 1 \{ t < 0.25 \} {λ}_{a u x} {L}_{a u x} \qquad ( 10 )$$

### Experiments

### SO(3) Synthetic Data

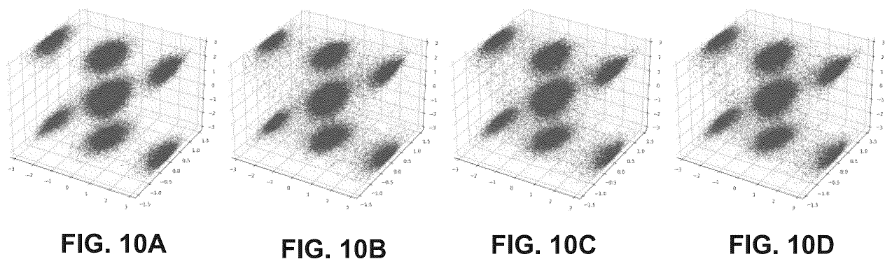

The FOLDFLOW models were evaluated on synthetic multimodal densities on SO(3). To accurately model this dataset, the method was restricted to the SO(3) component only. Quantitative results were reported by computing the Wasserstein distance between generated and ground truth samples in Table 1 and visualize the generated samples in FIGS. 10A-10D. All proposed methods correctly modeled all the modes of the ground truth distribution. However, FOLDFLOW exhibits mode shrinkage in relation to the ground truth. FOLDFLOW-OT, FOLDFLOW-SFM, and the simulated SDE results in comparable performance, with the OT-based method exhibiting the lowest Wasserstein distance. Importantly, this experiment shows that simulation-free approximation of the SDE does not hinder model performance, and combined with its significant speedup justifies its use in protein experiments.

TABLE 1
Mean and std of the 1- and 2-Wasserstein distances, computed
against 5000 points in the test set, over 5 seeds.

| (μ + σ) | W₁(x10⁻²) | W₂(x10⁻¹) |
|---|---|---|
| FOLDFLOW-BASE | 5.39 ± 0.88 | 1.52 ± 0.27 |
| FOLDFLOW-OT | 4.96 ± 0.27 | 1.25 ± 0.12 |
| FOLDFLOW-SFM | 4.92 ± 1.56 | 1.26 ± 0.49 |
| Simulated SDE | 5.13 ± 1.36 | 1.33 ± 0.44 |

### Protein Backbone Design

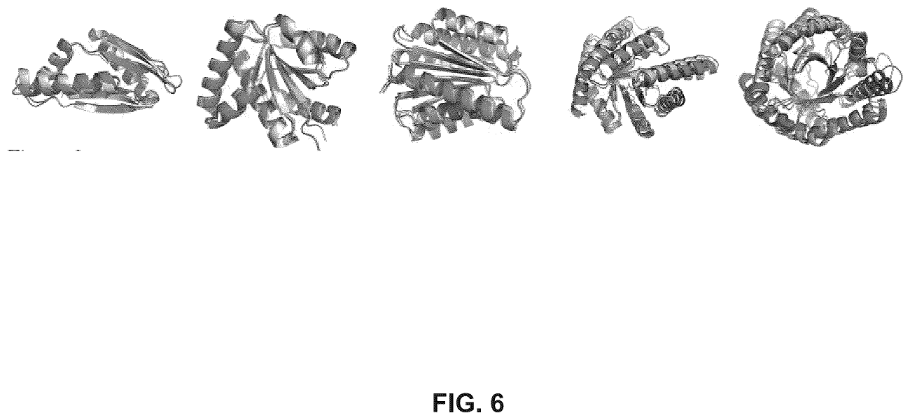

Next, FOLDFLOW models were evaluated in generating valid, diverse, and novel backbones by training on a subset of the Protein Data Bank (PDB) with 22,248 proteins. FOLDFLOW were compared to pretrained versions of FrameDiff (Yim et al., 2023b), both the published version (FrameDiff-ICML), and the improved version on the authors' GitHub (FrameDiff-Improved), Genie (Lin & AlQuraishi, 2023), and RFDiffusion, which is the gold standard (Watson et al., 2023). FrameDiff (FrameDiff-ICML-Retrained) was retrained on a dataset with 10% more admissible structures. Briefly, designability was considered to be the primary metric. This is because secondary metrics like diversity/novelty are intuitively only evaluated on designable proteins; for models that have low designability, this may result in a larger variance of diversity/novelty. A visualization of generated samples along with ESM-refolded structures are shown in FIG. 6.

FOLDFLOW models and baselines were evaluated across three established metrics in designability, diversity, and novelty of the generated samples. Table 2 demonstrates the results. FOLDFLOW outperforms FrameDiff-ICML-Retrained on all three metrics. FrameDiff, and its variants, are the most comparable baseline as it is the current SOTA model that does not utilize pre-training while using comparable resources. In contrast to FOLDFLOW, RFDiffusion uses a pre-trained backbone, a significantly larger model (60m vs. 17m parameters), a larger and different training set, and compute resources (1800 vs. 10 GPU days). Genie is trained on a larger dataset (195 k vs. 22 k), which hinders rigorous comparisons with FOLDFLOW. In the following, the performance of FOLDFLOW on each metric is analyzed in detail.

TABLE 2
Comparison of Designability (scRMSD < 2.0 Å), Diversity (avg. pairwise TMscore),
and Novelty (max. TM-score to PDB) as well as the training speed in steps/second.

| | Designability (↑) | Diversity (↓) | Novelty (↓) | iters/sec (↑) |
|---|---|---|---|---|
| RFDiffusion | 0.969 | 0.256 | 0.449 | — |
| Genie | 0.581 | 0.228 | 0.434 | — |
| FrameDiff-ICML | 0.402 | 0.237 | 0.542 | — |
| FrameDiff-Improved | 0.555 | 0.278 | 0.457 | — |
| FrameDiff-ICML-Retrained | 0.612 | 0.403 | 0.684 | 1.278 |
| FOLDFLOW-BASE | 0.657 | 0.264 | 0.452 | 2.674 |
| FOLDFLOW-OT | 0.820 | 0.247 | 0.460 | 2.673 |
| FOLDFLOW-SFM | 0.676 | 0.262 | 0.422 | 2.613 |

Designability. It has been empirically demonstrated that a protein backbone's experimental correctness is highly correlated to findability of a sequence that can be independently folded by other algorithms into the same structure. This is known as self-consistency and it is the chief metric used to test designability. First, 50 proteins were generated at lengths 100, 150, 200, 250, 300. ProteinMPNN (Dauparas et al., 2022) was applied at temperature 0.1 to the generated structures to acquire eight sequences associated with each structure. These sequences were refolded with ESMFold (Lin et al., 2022) to generate structures from these sequences. Finally, the FOLDFLOW generated structure was compared with the ESMFold generated structure from each sequence using the Ca-RMSD (scRMSD) metric. A protein was considered designable if scRMSD <2.0 Å and measure the fraction of generated proteins that achieve this threshold for at least one sequence (higher is better).

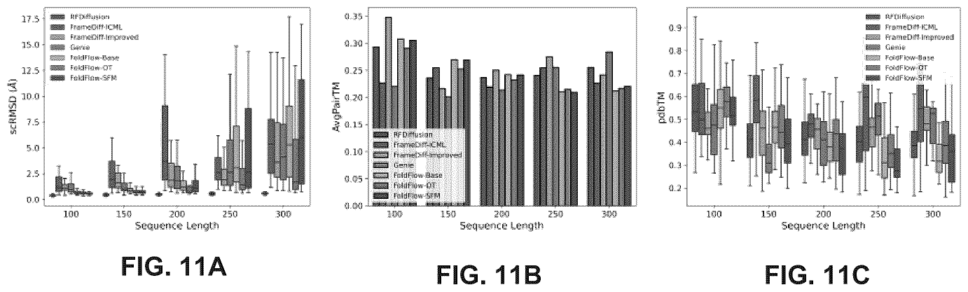

As shown in Table 2, all FOLDFLOW models achieve significantly higher designability scores than all FrameDiff models, and appreciably close the gap to RFDiffusion, e.g Δ=0.149 vs Δ=0.357 for FOLDFLOW-OT FrameDiff-ICML-Retrained respectively. When retrained on the dataset with 10% more samples, FrameDiff was more designable, but was less diverse and novel. While FOLDFLOW-OT created the highest fraction of designable proteins (excluding RFDiffusion), it had relatively low diversity and novelty. Adding stochasticity with FOLDFLOW-SFM resulted in a model that beats FrameDiff-Improved on every metric and dramatically improved novelty at the cost of worse designability (Table 2). In FIGS. 11A-11C, designability was plotted versus sequence length and the largest gains on sequence lengths <300 was observed.

Diversity. The average pairwise TM-score of the designable generated samples averaged across lengths was used as the diversity metric (lower is better). An inverse correlation was observed between designability and diversity. FOLDFLOW models have comparable diversity to the baselines with FOLDFLOW-OT being the most diverse. Specifically, the models improved over FrameDiff-Improved and RFDiffusion but were worse than FrameDiff-ICML and Genie, but had better designability which is of primary importance.

Novelty. Designing novel but realistic protein structures compared to the training data was also an important goal. Novelty was measured using the minimum TM-score of designable generated proteins to the training data (lower is better). FOLDFLOW-SFM designed the most novel structures against all methods including RFDiffusion and Genie. The coupling of high designability and high novelty was a strength of the FOLDFLOW models, and in particular FOLDFLOW-SFM.

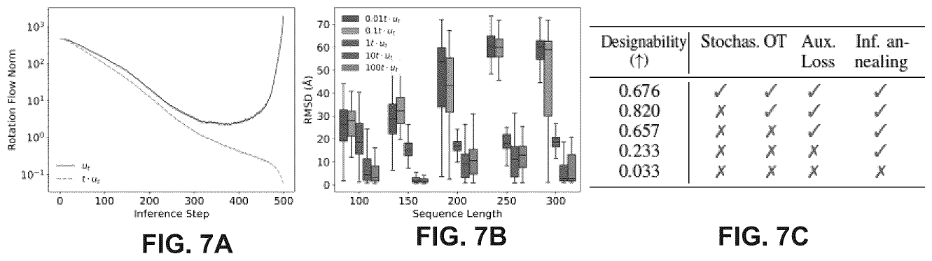

Inference Annealing refers to a numerical strategy during inference that greatly improves the designability of FOLDFLOW. Instead of following the theoretical ODE or SDE for generating rotations, a multiplicative scaling of the velocity was used—e.g. dRₜ=i(t)ν_{θ}(t, Rₜ)dt+γ(t)dBₜ for some positive function i(t). In practice, i(t)=ct for some constant c. This annealing removed an unwanted increase in the flow norm during the end of inference FIG. 7A. A larger c drastically increased the designability of FOLDFLOW. In practice, values of c≈10 (FIG. 7B) led to designable yet diverse structures.

Ablation Study of FOLDFLOW. Various additions to the FOLDFLOW-BASE model were ablated, as shown in FIG. 7C. OT without stochasticity created the most designable model, but adding stochasticity helped increase novelty and diversity. Inference annealing improved the performance of the model in terms of achieving higher designability.

### Equilibrium Conformation Generation

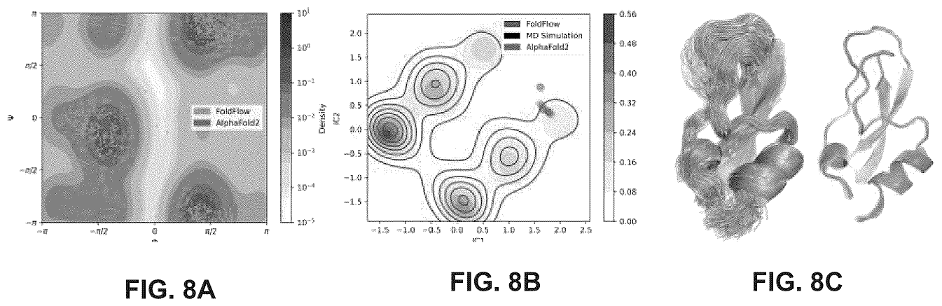

Proteins are not static objects; they naturally exist in an equilibrium of conformations. Modelling various protein conformations is valuable in determining biological behaviours such as mechanisms of actions or binding affinity to other proteins. Unlike diffusion models, FOLDFLOW can easily be instantiated from any sampleable source distribution. To test this, the equilibrium distribution of a protein was modeled given initial predicted structures from pre-trained folding models including OmegaFold (Wu et al., 2022b), and ESMFold (Lin et al., 2022). The training target distribution consisted of 200,000 frames at 5 ns intervals of a 1 ms molecular dynamics trajectory of the BPTI protein (Shaw et al., 2010); the inference of FOLDFLOW was tested against 20,000 unseen frames in that trajectory. FOLD-FLOW can successfully model both the general set of conformations, as indicated by ICA of the dihedral angles in FIG. 8B, as well as the highly flexible residues, as seen in the 2D Ramachandran plot in FIG. 8A. This approach can capture all the modes of distribution in contrast to AlphaFold2, which does not model the flexibility well (FIG. 8C). Finally, the ability to learn from any source distribution and not just an uninformative prior is a key advantage offered by FOLDFLOW enabling straightforward application to this task which is not possible using a diffusion model.

### Additional Methods and Analysis

SO(3) LIE GROUP The Special Orthogonal group in 3 dimensions, SO(3) consists of the 3D rotation matrices:

$$S O ( 3 ) = \{ r ∈ : {r}^{T} r = r {r}^{T} = I , det r = 1 \} \qquad ( 11 )$$

It is a matrix lie group with the lie algebra given by:

$$S O ( 3 ) = \{ r ∈ : {r}^{T} = - r \} \qquad ( 12 )$$

### Parametrizations of SO(3).

The skew-symmetric matrices r∈SO(3) can be identified with a vector ω∈ [symbol] such that ∀∀∈ [symbol] , rv=ω×ν, where × indicates the cross product. This vector is known as the rotation vector. The magnitude of this vector, ω=∥ω∥ is the angle of rotation its direction, eω=ω/∥ω∥ is the axis of rotation.

Mapping the [symbol] vector to the skew-symmetric matrix is known as the hat operation, (\hat{.}). Another parametrization of SO(3) is with Euler angles, described using three angles (ϕ, θ, ψ). A common convention is to use the x-convention, where the rotation is given by: a rotation about the z-axis by ϕ, a second rotation about the former x-axis by θ, and a last one about the former z-axis by ψ.

Metric on SO(3). First, recall that a metric is a bilinear function [symbol] , [symbol] : [symbol] × [symbol] → [symbol] that is both symmetric and positive definite. Additionally, recall that a quadratic form on a manifold M is a bilinear map TxM×TxM→ [symbol] that is smooth and symmetric. A positive-definite quadratic form is, therefore, a metric. Consider the following symmetric positive definite quadratic form defined:

$$Q = ( \begin{matrix}A & {B}^{T} \\ B & C\end{matrix} ) \qquad ( 13 )$$

A canonical choice for the metric of SO(3) is obtained by taking Q=1/2I, resulting in a bi-invariant metric on SO(3). Therefore, the metric is given by:

$${〈 {r}_{1} , {r}_{2} 〉}_{S O ( 3 )} = t r ( {r}_{1}^{T} Q {r}_{2} ) = \frac{1}{2} t r ( {r}_{1}^{T} {r}_{2} ) \qquad ( 14 )$$

Note that the inner product on Lie groups consumes elements of the Lie algebra and, because the left action is transitive, this inner product is well-defined for all tangent spaces of the group elements.
The Distance Induced by this Metric is Given by:

$${d}_{S O ( 3 )} ( {r}_{1} , {r}_{2} ) = ‖ log ( {r}_{1}^{T} {r}_{2} ) ‖ F \qquad ( 15 )$$

for r₁, r₂ ∈SO(3) and where the Frobenius matrix norm is used.

The exponential and logarithmic maps on SO(3). Generally speaking, the exponential and logarithmic maps of a Lie group G relate the elements in the group to the lie algebra, G. In the case of matrix Lie groups, these coincide with the matrix exponential:

$$g = exp ( 𝔤 ) = {\sum }_{n = 0}^{N} \frac{1}{n !} {𝔤}^{n} \qquad ( 16 )$$

and the matrix logarithm:

$$𝔤 = log ( g ) = {\sum }_{n = 0}^{N} \frac{{( - 1 )}^{n - 1}}{n} {( g - I )}^{n} \qquad ( 17 )$$

For SO(3), since the elements of the lie algebra are skew-symmetric matrices, eq. (16) for the matrix exponential can be simplified significantly to obtain a closed-form, known as Rodrigues formula. Given ω a rotation vector, and ω\hat{ }∈ so(3), the corresponding element of the lie group, r∈SO(3) is given by:

$$r = exp \hat{ω} = cos ( ω ) I + sin ( ω ) {e}_{ω} + ( 1 - cos ( ω ) ) {e}_{ω} {e}_{ω}^{1} \qquad ( 18 )$$

where ω and e_{ω} are the angle and axis of rotation for w.

Similarly, the matrix logarithm can be expressed using the rotation angle:

$$log ( ) = \{ \begin{matrix}\frac{ω}{2 sin ( ω )} ( r - {r}^{T} ) & if r \neq I , \\ 0 & if r = I\end{matrix} . \qquad ( 19 )$$

### SE(3) Lie Group

The special Euclidean group, SE(3) is used to represent rigid body transformations in 3 dimensions:

$$SE ( 3 ) = \{ ( \begin{matrix}r & s \\ 0 & 1\end{matrix} ) r ∈ SO ( 3 ) , s ∈ ( , + \} \qquad ( 20 )$$

Represented by this 4×4 matrix and with the group operation defined by matrix multiplication, this group can be seen as a subgroup of the general linear group GL(4, [symbol] ). The lie algebra of the group se(3) is given by:

$$se ( 3 ) = \{ r = ( \begin{matrix}r & s \\ 0 & 0\end{matrix} ) : r ∈ so ( 3 ) , s ∈ \} \qquad ( 21 )$$

Note that the tangent space of [symbol] is isomorphic to the space itself. This lie algebra is isomorphic to [symbol] using the map: r→(ω, s), where the skew-symmetric matrix r∈ [symbol] (3) with its axis-angle representation, ω∈ [symbol] . As the group of translations, ( [symbol] , +) is a normal subgroup of SE(3), the group can be understood as a semi-direct product: SE(3)=SO(3) [symbol] ( [symbol] , +).

Metric on SE(3). Although there are many possible choices for metrics on SE(3), none of them are bi-invariant. Instead, one can choose to build a left-invariant or right-invariant metric. A simple choice for the quadratic form Q from eq. (13) is setting the matrices A=C=I₃ and B=0 (Park & Brockett, 1994), which gives:

$$Q = ( \begin{matrix}{I}_{3} & 0 \\ 0 & {I}_{3}\end{matrix} ) \qquad ( 22 )$$

Using this metric define an inner product on SE(3) as

$$〈 {r}_{1} , {r}_{2} 〉 SE ( 3 ) = tr ( r \frac{T}{1} {Qr}_{2} ) ,$$

where tr is the trace operation. Writing out the inner product explicitly for r₁, r₂ ∈ [symbol] (3),

$$tr ( {r}_{1}^{T} {Qr}_{2} ) = tr ( \begin{matrix}{r}_{1}^{1} {r}_{2} & {r}_{1}^{1} {s}_{2} \\ {s}_{1}^{T} {r}_{2} & {s}_{1}^{T} {s}_{2}\end{matrix} ) \qquad ( 23 )$$

This means that the geodesics on SE(3) can be obtained from the geodesics on the product manifold SO(3)× [symbol] :

$${d}_{SE ( 3 )} ( {x}_{1} , {x}_{2} ) = \sqrt{{{d}_{SO ( 3 )} ( {r}_{1} , {r}_{2} )}^{2} + {{d}_{{ℝ}^{3}} ( {s}_{1} , {s}_{2} )}^{2}} \qquad ( 25 )$$

where x₁=(r₁, s₁), x₂=(r₂, s₂)∈SE(3), dₛ₀(3) is defined in eq. (15) and [symbol] is the usual Euclidean distance.
The Isotropic Gaussian Distribution on so(3)

IGSO(3) density. The isotropic Gaussian distribution on SO(3) is parametrized by a mean, r∈SO(3) and a concentration parameter, ∈∈R. It can be parametrized in axis-angle, where the axis of rotation is sampled uniformly and the angle of rotation ω has probability density function (pdf) given by:

$$f ( {ω}_{x} , ϵ ) = {\sum }_{l = 0}^{∞} ( 2 l + 1 ) {e}^{- l ( l + 1 ) e} \frac{sin ( ( l + \frac{1}{2} ) {ω}_{x} )}{sin ( \frac{{ω}_{x}}{2} )} \qquad ( 26 )$$

Although this expression contains an infinite sum, Matthies et al. (1900) has shown that for ∈≤1, it can be approximated by a closed-form equation:

$$f ( {ω}_{x} , ϵ ) = \sqrt{π} {ϵ}^{- \frac{3}{2}} {e}^{\frac{ϵ - \frac{{ω}^{2}}{ϵ}}{4}} \frac{( ω - {e}^{- \frac{{π}^{2}}{ϵ}} ( ( ω - 2 π ) {e}^{\frac{π ω}{ϵ}} + ( ω + 2 π ) {e}^{- \frac{π ω}{ϵ}} ) )}{2 sin \frac{ω}{2}} \qquad ( 27 )$$

Sampling from IGSO(3). Sampling from IGSO(3) was done following Leach et al. (2022). The angle of rotation was obtained by inverse transform sampling, where the cumulative density function was approximated using the pdf above, scaled by uniform density on SO(3) with density f(ω)=1-cos ωπ; the axis was sampled uniformly from S². The closed-form approximation of eq. (27) makes the computation of the cdf, and hence the sampling process very efficient.

Flow Matching In

[symbol]

To perform FOLDFLOW on SE(3), two different flows were considered. One on SO(3) that was described above and another one on [symbol] that is described herein depending on the consider FOLDFLOW method. Riemannian Flow Matching is a generalization of Flow Matching on Riemannian manifold. This means that the objective is also to regress a conditional vector field built from conditional probability paths. Described in this section, are conditional probability paths and conditional vector fields that were used respectively by Lipman et al. (2022) and Tong et al. (2023b). The main difference is that the conditional probability path is now a Gaussian conditioned on a latent variable z˜q(z) with variance σt, pt(s)=N(s|z, σt). The conditional vector field has a closed form derived from the following Theorem:

Theorem 1 (Theorem 3 of Lipman et al. (2022)). The unique vector field whose integration map satisfies ρₜ(s)=μₜ+σₜs has the form

$${μ}_{t} ( s ) = \frac{{σ}_{t}^{′}}{{σ}_{t}} ( s - {μ}_{t} ) + {μ}_{t}^{′} \qquad ( 28 )$$

Flow Matching. In the context of data living in the Euclidean space [symbol] . Identifying the condition z with a single datapoint z=s₁, and choosing a smoothing constant σ>0, one sets

$${p}_{t} ( s \vert z ) = N ( s \vert {ts}_{1} , {( t σ - t + 1 )}^{2} ) , \qquad ( 29 )$$

$${μ}_{t} ( s \vert z ) = \frac{{s}_{1} - ( 1 - σ ) s}{1 - ( 1 - σ ) t} , \qquad ( 30 )$$

which is a probability path from the standard normal distribution (p₀(x|z)=N (x; 0,1)) to a Gaussian distribution centered at X₁ with standard deviation σ(p₁ (x|z)=N (x; x₁, σ²). If one sets q(z)=q(x₁) to be the uniform distribution over the training dataset, the objective introduced by Lipman et al. is equivalent to the CFM objective (1) for this conditional probability path.

OT-Conditional Flow Matching. As explained in the main paper, the probability path used in FM is not the optimal transport probability paths between the distributions ρ₀ and ρ₁. Therefore, straighter flows are desirable for faster inference and more stable training. To achieve that, leverage the optimal transport theory to ensure the probability path involves the Euclidean McCann interpolants defined as ρₜ=tΨ(s₀)+(1−t) so. However, as the map Ψ is intractable in practice, rely on the Brenier theorem which makes a connection between the map Ψ and the optimal transport plan π. Therefore set the mean of Gaussian conditional probability path as μₜ=ts₁+(1−t) sp and the latent distribution q(S₀, S₁)=π(S₀, S₁).

$${p}_{t} ( s \vert {s}_{0} , {s}_{1} ) = N ( s \vert {ts}_{1} + ( 1 - t ) {s}_{0} , {σ}^{2} ) , \qquad ( 31 )$$

$${p}_{t} ( s ) = \int N ( s \vert {ts}_{1} + ( 1 - t ) {s}_{0} , {σ}^{2} ) π ( {s}_{0} , {s}_{1} ) {ds}_{0} {ds}_{1} , \qquad ( 32 )$$

$${u}_{t} ( s \vert z ) = {s}_{1} - {s}_{0} . \qquad ( 33 )$$

In the case of the Euclidean space the FM loss is equal to [symbol] _{FOLDFLOW-} [symbol] =∥ν_{θ(t, s)}−Uₜ(s|z)∥. This can be simplified to down to [symbol] _{FOLDFLOW-} [symbol] =∥∇_{θ}(t, s)−s₁-S₀∥. This method is the main inspiration to develop FOLDFLOW-OT.

### Riemannian Optimal Transport

### Background on Riemannian Optimal Transport:

Optimal transport on Riemannian manifold was first studied in the seminal work of McCann (2001); refer to Villani (2003; 2008) for a review of all results. Recently, optimal transport has also drawn attention from the machine learning community. The (static) Kantorovich optimal transport problem seeks a mapping from one measure to another that minimizes a displacement cost. Formally, define the 2-Wasserstein distance between distributions ρ₀ and ρ₁ on M with respect to the cost

$$c ( x , y ) = \frac{1}{2} {d ( x , y )}^{2}$$

as:

$${W ( {ρ}_{0} , {ρ}_{1} )}_{2}^{2} = {inf}_{π ∈ \prod ( {ρ}_{0} , {ρ}_{1} )} {\int }_{{M}^{2}} c ( x , y ) d π ( x , y ) , \qquad ( 34 )$$

where Π(ρ₀, ρ₁) denotes the set of all joint probability measures on M×M whose marginals are ρ₀ and ρ₁. To compute the optimal transport plan, rely on the POT library Flamary et al. (2021). This problem is a relaxation of the well-known Monge formulation described in the main paper. The Monge optimal transport problem is defined as

$$OT ( {ρ}_{0} , {ρ}_{1} ) = {inf}_{Ψ : Ψ \neq {ρ}_{0} = {ρ}_{1}} {\int }_{{M}^{2}} c ( x , y ) d π ( x , y ) , \qquad ( 35 )$$

When M is a smooth compact manifold with no boundary and ρ0 has a density, (McCann, 2001, Proposition 9) shows that the map T exists and is unique. This is an extension to Riemannian manifold of the well-known Brenier Theorem (Brenier, 1991). The optimal transport map Y′ and the McCann interpolation have then the following form: . . .

$$Ψ ( x ) = {exp}_{x} ( - ∇ ϕ ( x ) ) , \qquad ( 36 )$$

$${Ψ}_{i} ( x ) = {exp}_{x} ( - t ∇ ϕ ( x ) ) .$$

where ϕ is a c-concave function. Furthermore, the optimal transport plan is supported on the graph of the Monge map, i.e., π=(id, Ψ) #ρ0. Therefore, knowing the transport plan leads to the Monge map.

Minibatch OT approximation. For empirical distributions, the Kantorovich problem is a linear program and can be efficiently solved with the simplex algorithm. However, when dealing with large datasets, computing and storing the transport plan π for Optimal Transport (OT) can be challenging due to its cubic time and quadratic memory complexity with respect to the number of samples. To address this, a minibatch OT approximation is often employed. While this approach introduces some error compared to the exact OT solution (Fatras et al., 2020), it has been proven effective in various applications such as domain adaptation and generative modeling (Damodaran et al., 2018; Genevay et al., 2018). Specifically, during training, for each source and target minibatch, pairs of points are sampled from the optimal transport plan computed between the pair (x, y)˜πbatch. The batch size can be small compared to the full dataset size and still give a good performance, which aligns with prior studies (Fatras et al., 2021b;a). This strategy is also at the heart of the OT-CFM methods (Tong et al., 2023b;a; Pooladian et al., 2023).

Proposition 1. Consider SE(3)^{N}₀ with the product distance

$${d}_{{SE ( 3 )}_{0}^{N}}$$

and two completely supported probability distributions ρ₀,

$${ρ}_{1} ∈ ℙ ( {SE ( 3 )}_{0}^{N} ) .$$

In addition, suppose that ρ₀ is absolutely continuous with respect to Riemannian volume form (i.e., p₀<<dx). Then for the distance

$$c = \frac{1}{2} {d}_{{SE ( 3 )}_{0}^{N}}^{2} ,$$

tne Kantorovien ana ivionge problems admit a unique solution that is connected as follows π=(id×ψ)#ρ₀, where ψ is almost uniquely determined everywhere ρ₀. Furthermore, we have that ψ(x)=expₓ (∇ϕ(x)) for some

$${d}_{{SE ( 3 )}_{0}^{N -}}^{2}$$

concave function ϕ.

Proof. The manifold SE(3) is a connected, complete, (C^{∞}) smooth manifold without boundary. SE(3)^{N} (equipped with the usual product distance) is a finite Cartesian product of connected, complete, smooth manifolds without boundary and therefore it is itself connected, complete, smooth manifold without boundary. Check these assumptions are also satisfied by SE(3)^{N} by noting that SE(3)^{N} can be written as N-1 copies of SE(3) where the R³ component is mean subtracted—i.e.

$${s}^{n} = s - 1 / N {\sum }_{i = 1}^{N} \text{?} ,$$

$$\text{?} \text{indicates text missing or illegible when filed}$$

and the final Nth element in the product is the mean,

$$1 / N {\sum }_{i = 1}^{N} {s}_{i} \text{?} .$$

$$\text{?} \text{indicates text missing or illegible when filed}$$

Certainly, the first N-1 components satisfy connectedness, and completeness, and are manifolds that are smooth without boundary. Furthermore, the disintegration of measures on SE(3)^{N} (Proposition 3.5 (Yim et al., 2023b)) allows us to define a measure u proportional to R³ for the final component Nth component. Therefore, by the assumptions on the measures ρ₀, ρ₁, apply the following Theorem from Villani (2003) to get the desired results.

Theorem 2 (Theorem 2.47, Villani (2003)). Let M be a connected, complete and smooth (C3) Riemannian manifold without boundary, equipped with its standard volume measure. Let p0, p1 be two compactly supported distributions and set the ground cost c(x, y)=1 2d(x, y)2 with d the geodesic distance on M. Further, assume that p0 is absolutely continuous with respect to the volume measure on M. Then the Kantorovich and Monge problems admit a unique solution that is connected as follows π=(id×ψ)=p0: where Ψ is almost uniquely determined everywhere p0. Furthermore ψ(x)=expₓ(∇ϕ(x)) for some d2-concave function ϕ.

### Stochastic Riemannian Flow Matching

Follow the presentation in Jensen et al. (2022) to define the Brownian bridge on a Lie group G endowed with a metric. Note that log is the inverse of the Riemannian exponential map. However, if the metric is bi-invariant, which is the case for SO(3), it coincides with the Lie group logarithm. Simulate a bridge on G via the guided diffusion SDE (using. for the Stratonovich integral), for a process conditioned to reach ν at t=1.

$$d {B}_{t} = - \frac{1}{2} V \text{?} ( {B}_{t} ) dt + {V}_{i} ( {R}_{t} ) \text{?} ( D {B}_{t}^{i} - \frac{{{log}_{{R}_{t}} ( v )}^{i}}{1 - t} dt ) {R}_{0} = {r}_{0} , \qquad ( 37 )$$

$$\text{?} \text{indicates text missing or illegible when filed}$$

where Vᵢ (xr=(dLᵣ)ₑ νi_{with} {ν₁, . . . , ∇_{d}} an orthonormal basis of TₑG, and where Bₜ is a Brownian motion of G. On SO(3), since the metric is bi-invariant, we have Vo=0. In this work, model the guided bridge with a diffusion that does not depend on the process Rt. In this case, the Stratonovich and Itô formulations are the same, yielding the reversed process defined in Eq. 7.

### Simulation-Free Approximation of Brownian Bridges on SO(3)

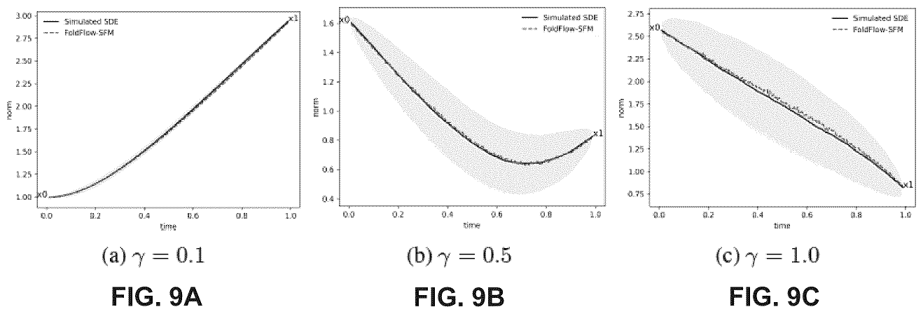

Next, the fidelity of the simulation-free SDE employed in FOLDFLOW-SFM was numerically investigated in relation to the guided drift SDE in eq. (8). FIGS. 9A-9C plot the mean and the standard deviation (over 1024 data points) of the distribution of the SO(3)-norm along the trajectory against time, for three different values of the diffusion coefficient, γ. The true simulated Brownian bridge (bold black line) was in close proximity to the simulation-free FOLDFLOW-SFM SDE (red dotted lines). Furthermore, this holds for the entire trajectory and leads to overlapping shaded regions that correspond to the standard deviation of the norm. This result adds empirical substantiation to using the FOLDFLOW-SFM as a drop-in and fast approximation to the Brownian bridge SDE on SO(3).

Proposition 2. Given

$${ρ}_{t} ( x ) > 0 , ∀ x ∈ {SE ( 3 )}_{0}^{N} ,$$

the conditional and unconditional FOLDFLOW-SFM losses have equal gradients w.r.t. θ: ∇θ [symbol] _{USFM} (θ)=∇_{θ} [symbol] _{SFM} (θ).

$$Proof . Let {u}_{t} = {ρ ( z )}_{} ⌊ \frac{{ρ}_{t} ( x \vert z )}{{ρ}_{t} ( x )} {u}_{t} ( x \vert z ) ⌋ , for x ∈ {SE ( 3 )}_{0}^{N} . Claim that : \\ ∇ \text{?} {z ∼ ρ ( z ) , x ∼ {ρ}_{t} ( x \vert z )}_{} [ {‖ \text{?} ( t , x ) - {u}_{t} ( x \vert z ) ‖}_{{SE ( 3 )}_{0}^{N}}^{2} ] = ∇ \text{?} {x ∼ {ρ}_{t ( x )}}_{} [ {‖ \text{?} ( t , x ) - {u}_{t} ( x ) ‖}_{{SE ( 3 )}_{0}^{N}}^{2} ] \qquad ( 38 )$$

$$\text{?} \text{indicates text missing or illegible when filed}$$

From disintegration of measures (Pollard (2002) and Proposition 3.5 form Yim et al. (2023b)), we know the probabilities ρₜ (x)√ρₜ (r₁) . . . .ρₜ (r^{N}) ρₜ (s¹) . . . .ρₜ (S^{N}), and similar for the conditional probability ρₜ (x|z). Given that by eq. (24), the metric on SE(3) also factorizes into metric on SO(3) and [symbol] , it suffices to prove the claim for SO(3) and [symbol] . The claim can therefore be stated as follows, where r^{l} is written as r and s^{l} is written as s for conciseness.

$$∇ \text{?} {z ∼ ρ ( z ) , x ∼ {ρ}_{t} ( r \vert {z}_{r} )}_{} [ {‖ \text{?} ( t , r ) - {u}_{t} ( r \vert {z}_{r} ) ‖}_{SO ( 3 )}^{2} ] = ∇ \text{?} {r ∼ {ρ}_{t} ( r )}_{} [ {‖ \text{?} ( t , r ) - {u}_{t} ( r ) ‖}_{SO ( 3 )}^{2} ] \qquad ( 39 )$$

$$∇ \text{?} {z ∼ ρ ( z ) , s ∼ {ρ}_{t} ( s \vert {z}_{s} )}_{} [ {‖ \text{?} ( t , s ) - {u}_{t} ( s \vert {z}_{s} ) ‖}_{{ℝ}^{3}}^{2} ] = ∇ \text{?} {s ∼ {ρ}_{t} ( s )}_{} [ {‖ \text{?} ( t , s ) - {u}_{t} ( s ) ‖}_{{ℝ}^{3}}^{2} ] \qquad ( 40 )$$

$$\text{?} \text{indicates text missing or illegible when filed}$$

The proof of this claim follows a similar structure to Chen & Lipman (2023). Next, prove eq. (40). Dropping the distributions for conciseness:

$$∇ \text{?} ( \text{?} [ {‖ \text{?} ( t , r ) - {u}_{t} ( r \vert {z}_{r} ) ‖}^{2} ] - \text{?} [ {‖ \text{?} ( t , r ) - {u}_{t} ( r ) ‖}^{2} ] ) = ∇ \text{?} ( - 2 \text{?} {〈 \text{?} ( t , r ) - {u}_{t} ( r \vert {z}_{r} ) 〉}_{SO ( 3 )} - 2 {r}_{} {〈 \text{?} ( t , r ) , {u}_{t} ( r ) 〉}_{SO ( 3 )} ) \qquad ( 41 )$$

$$\text{?} \text{indicates text missing or illegible when filed}$$

### Now:

$$( 42 )$$

$$\begin{matrix}{r}_{} ( \text{?} ( t , r ) , {u}_{t} ( r ) ) = {\int }_{0}^{1} {\int }_{SO ( 3 )} {〈 \text{?} ( t , r ) , {u}_{t} ( r ) 〉}_{SO ( 3 )} {ρ}_{t} ( r ) d {vol}_{r} \\ = {\int }_{0}^{1} 〈 \text{?} ( t , r ) {\int }_{SO ( 3 )} \frac{{ρ}_{t} ( r \vert {z}_{r} )}{{ρ}_{t} ( r )} {u}_{t} ( r \vert {z}_{r} ) ρ ( {z}_{r} ) d {vol}_{{z}_{r}} 〉 {ρ}_{t} ( r ) {d}_{r} \\ = {\int }_{0}^{1} {\int }_{SO ( 3 )} 〈 \text{?} ( t , r ) , {u}_{t} ( r \vert {r}_{z} ) ) {ρ}_{t} ( r \vert {r}_{z} ) ρ ( {r}_{z} ) d {vol}_{r} {dvol}_{{z}_{r}} \\ = {r , {z}_{r}}_{} 〈 \text{?} ( t , r ) {u}_{t} ( r \vert {z}_{r} ) 〉\end{matrix}$$

$$\text{?} \text{indicates text missing or illegible when filed}$$

### Toy Model Parameterization

For the vector-field parametrization, the goal is to create a function that by construction lies on the tangent space of the manifold. For the toy experiments, this is done by using a 3-layer MLP, and projecting the output of the network to the tangent space of the input. That is, similar to Chen & Lipman (2023), we have:

$$\text{?} ( t , r ) = {II}_{r} MLP ( t , r ) , \qquad ( 43 )$$

$$\text{?} \text{indicates text missing or illegible when filed}$$

where II_{T}(M) projects a 3×3 matrix onto [symbol] SO(3). This operation essentially computes the skey-symmetric component of M, given by

$$\frac{M - {M}^{T}}{2}$$

and parallel transports it to the tangent space of r using left matriz multiplication which is the group operation SO(3).

Here, the qualitative results of the toy experiments are presented. In FIGS. 10A-10D, all the three models, FOLDFLOW-BASE, FOLDFLOW-OT FOLDFLOW-SFM learn to correctly model the modes of the ground-truth distribution with a slight model shrinkage in the FOLDFLOW-BASE.

### Protein Backbone Generation Experiment Additional Results

FIG. 13 depicts five proteins generated by FOLDFLOW-SFM from each backbone length. Here, the generated structure is shown in green and the best ESM-refolded structure out of eight sequences generated using ProteinMPNN. FOLDFLOW-SFM generates diverse folds that refold with diversity in secondary structure and overall 3D conformation.

FIGS. 11A-11C show the comparison of the performance of models on designability, diversity, and novelty tasks for different backbone lengths. In particular, FOLDFLOW closes the gap between models without pretraining (Genie, FrameDiff, FOLDFLOW) and RFDiffusion in terms of designability, particularly on shorter sequences (e.g., less than 200).

Notably, there is a trade-off between designability and diversity/novelty, both at the short sequence lengths and as sequence length increases. For longer sequences (250, 300), while FOLDFLOW models are comparable in terms of designability, they generate significantly more diverse and novel structures as compared to all other models, even RFDiffusion (although RFDiffusion still generates significantly more designable proteins at these lengths.

### Timing Comparison

Here, the number of steps per second for each model was compared, where a step corresponds to a forward and backwards pass on the effective batch size as defined in Equation (46) on a single GPU. Here FOLDFLOW is over 2× faster than FoldFlow-Improved per step. This drastic improvement is due to a number of optimizations, with the largest being that it avoids the costly IGSO(3) score computation which is necessary for their method. The model was trained in Pytorch using distributed data-parallel (DDP) across four NVIDIA A100-80 GB GPUs for roughly 2.5 days. This is substantially less than comparable models (table 3). RFDiffusion requires the use of pre-trained weights from RosettaFold which trained for 4 weeks on 64 V100 GPUs (Watson et al., 2023).

TABLE 3
Training resources for protein generation models.

| Model | Training time | | Optimization Steps | | #gpus | Distributed training |
|---|---|---|---|---|---|---|
| RFDiffusion | 28 + 3 | days | — | | 64 + 8 | — |
| RFDiffudion w/o pretraining | 3 | days | — | | 8 | — |
| Genie (SwissProt) | ~8 | days | ~800 | k | 6 | DP |
| FrameDiff-ICML | ~7 | days | ~1.9 | m | 2 | DP |
| FrmaeDiff-Improved | +7 | days | +1.9 | m | 2 | DP |
| FOLDFLOW | ~2.5 | days | 600 | k | 4 | DDP |

### Equilibrium Conformation Generation Experiment

Proteins take on many different physical conformations in the real world. These conformations dictate many important attributes of a protein's behaviour, e.g., how one protein might bind to another. As a protein's conformations generally do not deviate greatly from one another, a desirable approach would be to start from a noised version of a known conformation of the protein to generate another conformation. FOLDFLOW was discovered as an ideal candidate for this setting. Its efficacy is shown in FIGS. 8A-8C.

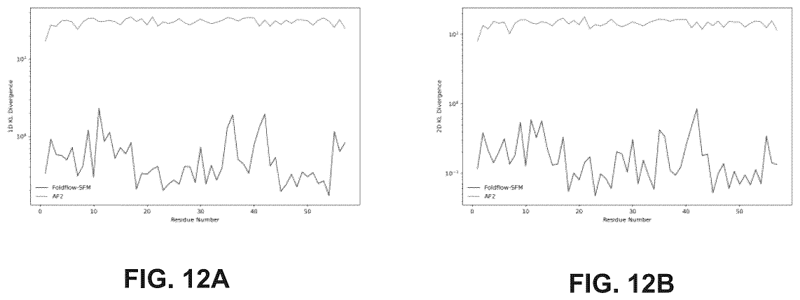

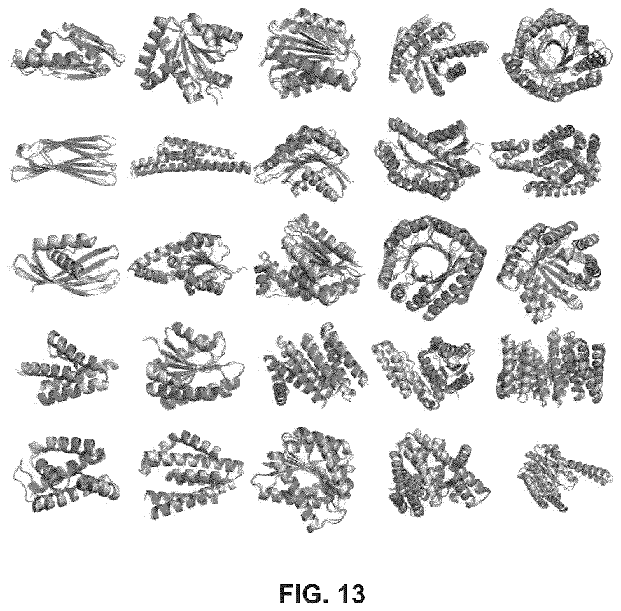

Bovine pancreatic trypsin inhibitor (BPTI) was chosen for studying in this experiment. The 58-residue protein is the first protein whose dynamics were studied experimentally by nuclear magnetic resonance (NMR) and was the first protein that was simulated by molecular dynamics for 1 ms. On timescales ranging from nanoseconds to milliseconds, the dynamics of BPTI involve protein backbone structural changes that, for example, accommodate water molecule exchange and disulfide isomerization (Persson & Halle, 2008). The 1-ms MD simulation trajectory was used at a temperature of 300 K (Shaw et al., 2010) to reproduce and interpret the kinetics of folded BPTI. To construct the source distribution, the four folded conformations from each of AlphaFold2, ESMFold, RoseTTAFold, and Unifold were first generated. The source distribution then added a small amount of noise from the standard Gaussian and IGSO(3). FOLDFLOW was trained for one day on 4 A100 GPUs. FOLDFLOW generates conformations covering all modes of the true conformation distribution. Moreover, different conformations of BPTI were sampled from AlphaFold2 and were plotted on the Ramachandran and ICA plots, observing while FOLDFLOW can capture all modes of the distribution AlphaFold2 only captures one. Further FIGS. 12A-12B show that the KL divergence between the distribution of angles generated by FOLDFLOW was low and uniform, conveying it learned the distribution of the target.

### Further Discussion of Foldflow and Related Models

Symmetries as an inductive bias for flow matching. Leveraging symmetries as an inductive bias in deep learning models (for example by data augmentation or design equivariant models) has been shown to improve data efficiency and lead to better generalization. In the context of flow matching for proteins, the goal is to learn the vector field generating the flow, which maps an invariant source to an invariant target distribution, guaranteeing the existence of an equivariant vector field (Kohler et al., 2020; Bose & Kobyzev, 2021). Therefore, one way to exploit this symmetry would be to parameterize the vector field with an equivariant network, taking as input the 3D coordinates of the protein. Alternatively, since protein backbones can be parametrized by elements of SE(3)^{N}, one can directly construct the vector field by taking an intrinsic perspective by using charts on the manifold and their coordinate system. In this case, as the vector field lies on the tangent space of SE(3) it is equivariant by construction. While the FOLDFLOW models use a similar setup to FrameDiff, described herein are a number of improvements that help to stabilize training and improve performance. Indeed the additions lead to improvements on all metrics over the FrameDiff-Improved model released on GitHub which substantially improves on the designability over FrameDiff-ICML.
1. A bug in the score calculation for rotations means that there is a stop gradient in the rotation score calculation and FrameDiff-ICML is not trained to match the rotation score, which makes its performance quite impressive given this limitation. This bug is fixed in the FrameDiff-Improved model which uses a different score calculation.
2. The dataloader was switched from sampling uniform over proteins in the dataset, to uniform over clusters, then uniform within clusters. This changes the distribution of proteins but overall increases diversity as there are many similar proteins in a small number of clusters.
3. The rotation loss was changed to use a separate axis and angle component to reduce variance in the loss.

### Implementation Details and Experimental Setup Training and Inference

To describe the precise algorithm for training FOLDFLOW models over distributions in SE(3)^{N}. The starting distribution in SE(3)^{N} is r₁˜Uₛ₀₍₃₎ i.e. uniform over rotations and s₁˜N(0, I).After centering (i.e. subtracting the mean) this distribution will be uniform over rotations and with translations distributed according to the centered normal

$$( {r}_{1} , {s}_{1}^{c} ) ∈ {SE ( 3 )}_{0}^{N} ,$$

with

$${s}_{1}^{c} ∼ {N}^{c} ( 0 , 1 ) .$$

In algorithm 1, slightly abuse the notation and denote the output of the rotation part as ∇_{θ} as ν_{θ}(t, rₜ) and similarly the translation part of ν_{θ} as ν_{θ}(t, sₜ). Do not include separate algorithms for FOLDFLOW-BASE and FOLDFLOW-OT as they are simple modifications to FOLDFLOW-SFM. Set γᵣ (t)=0 and γₛ (t)=0, then recover the FOLDFLOW-OT algorithm. If in addition one removes the resampling in lines 4 and 5 then recover the FOLDFLOW-BASE algorithm.

Algorithm 1 FOLDFLOW-SFM training on SE(3)

[symbol]

| 1: | Input: Source and target ρ [symbol] (x₁), ρ [symbol] (x [symbol] )flow network v [symbol] , and diffusion scaling γᵣ(t), γ [symbol] (t) | | |
|---|---|---|---|
| 2: | while Training do | | |
| 3: | \| | t, x₀, x₁ ~ [symbol] (0, 1), ρ₀, ρ₁ | |
| 4: | \| | [symbol] ← OT(x₀, x₁) | [symbol] OT resampling step to obtain FOLDFLOW-OT |
| 5: | \| | (r₀, s₀), (r₁, s₁) ~ [symbol] | |
| 6: | \| \| | ${s}_{0}^{c} , {s}_{1}^{c} ← {s}_{0} - \frac{1}{N} {\sum }_{i} {s}_{0}^{t} , {s}_{1} - \frac{1}{N} {\sum }_{i} {s}_{1}^{t}$ | $mean subtract : ( {s}_{0} \text{?} , {r}_{0} ) , ( {s}_{1} \text{?} , {r}_{1} ) ∈ {SE ( 3 )}^{N} \text{?}$ |
| 7: | \| | rₜ ← exp [symbol] (t log [symbol] (r₁)) | [symbol] geodesic interpolant from eq. (2) |
| 8: | \| \| | ${s}_{i} ← {ts}_{1}^{c} + ( 1 - t ) {s}_{0}^{c}$ | [symbol] Interpolant (Euclidean) |
| 9: | \| \| | ${\hat{r}}_{t} ∼ {𝒳𝒢}_{SO ( 3 )} ( {r}_{t} , {γ}_{r}^{2} ( t ) t ( 1 - t ) )$ | [symbol] simulation-free approximation from eq. (9) |
| 10: | \| \| | ${\hat{s}}_{t} ∼ 𝒩 ( {s}_{t} , {γ}_{s}^{2} ( t ) t ( 1 - t ) )$ | |

| | | |
|---|---|---|
| 11: | \| \| \| | ${ℒ}_{FOLDFLOW} ← {‖ {v}_{θ} ( t , {\hat{r}}_{t} ) - \frac{{log}_{{r}_{t}} ( {r}_{0} )}{t} ‖}_{SO ( 3 )}^{2} + {‖ {v}_{g} ( t , {\hat{s}}_{t} ) - {s}_{1}^{c} - {s}_{0}^{c} ‖}^{2}$ |
| 12: | └ | [symbol] ← Update( [symbol] , ∇ [symbol] [symbol] _{FOLDFLOW}) |

| | |
|---|---|
| 13: | return v [symbol] |

[symbol]

indicates data missing or illegible when filed

### Sde Training and Inference

This section outlines the training and inference algorithms for the SO(3) component of FOLDFLOW-SFM The training algorithm is detailed in algorithm 2 while the inference algorithm is provided in algorithm 3. Similar to the toy experiment, for the protein modelling case, the architecture is constructed such that the output vector lies on the tangent space. For proteins, use the FrameDiff architecture (Yim et al., 2023b) over SE(3)NO, which is based on the structure module of AlphaFold2 (Jumper et al., 2021) following the initial work on diffusion models with AF2-like architectures (Anand & Achim, 2022). As described above, this architecture we outputs a predicted \hat{x}₀, which can then deterministically transform into a vector located at the tangent space of xₜ. This transformation can be split into 2N components, the N [symbol] components, and the N, SO(3) components. For the SO(3) components, calculate

$${v}_{θ} ( t , {r}_{t} ) = \frac{{log}_{{r}_{t}} {\hat{r}}_{0}}{t} , \qquad ( 44 )$$

and for the [symbol] components after centering,

$${v}_{θ} ( t , {s}_{t} ) = ( \frac{{s}_{t} -}{t} ) - \frac{1}{N} {\sum }_{i}^{N} ( \frac{{s}_{t} -}{t} ) i , \qquad ( 45 )$$

where ( [symbol] , [symbol] )= [symbol] =W_{θ}(t, xₜ). For [symbol] , ν₀(t, sₜ) is clearly on the tangent space as [symbol] is isomorphic to tis won tangent space. This is because Euclidean space is a flat space. For SO(3), ∇_{θ}(t, rₜ) is also on the tangent space or rₜ due to the definition of the log map. Since all components of the product space

$${SE ( 3 )}_{0}^{N}$$

are on the tangent space, ν_{θ}(t, Xₜ) is on the tangent space of

$${SE ( 3 )}_{0}^{N} .$$

Algorithm 2 FOLDFLOW-SEM training on SO(3)

| 1: | Input: Source and target ρ₁, ρ₀, diffusion schedule γ(•), flow network v [symbol] | | |
|---|---|---|---|
| 2: | while Training do | | |
| 3: | \| | t, x₀, x₁ ~ [symbol] (0, 1), ρ₀, | |
| 4: | \| | [symbol] ← OT(x₀, x₁) | |
| 5: | \| | r₀, r₁ ~ [symbol] | |
| 6: | \| | rₜ ← exp [symbol] (t log [symbol] (r₁)) | [symbol] geodesic interpolant from eq. (2) |
| 7: | \| | [symbol] ~ [symbol] _{SO(3)} (r [symbol] , γ² (t)t(1 − t)) | [symbol] simulation-free approximation from eq. (9) |
| 8: | \| \| | ${u}_{t} ( {\tilde{r}}_{t} \vert {r}_{0} , {r}_{1} ) ← \frac{{log}_{{r}_{t}} ( {r}_{0} )}{t}$ | |
| 9: | \| \| | ${ℒ}_{FOLDFLOW \cdot SFM} ← {‖ {v}_{θ} ( t , {r}_{t} ) - {u}_{t} ( {\tilde{r}}_{t} \vert {r}_{0} , {r}_{1} ) ‖}_{SO ( 3 )}^{2}$ | |
| 10: | └ | [symbol] ← Update( [symbol] , ∇ [symbol] [symbol] _{FOLDFLOW-SFM}) | |

| | |
|---|---|
| 11: | return v [symbol] |

[symbol]

indicates data missing or illegible when filed

Algorithm 3 FoldFlow-SFM Inference

| 1: | Input: Source distribution ρ₁, flow network v [symbol] , diffusion schedule γ(•), | | |
|---|---|---|---|
| | inference annealing i(•), noise scale, ζ, integration step size Δt. | | |
| 2: | Sample r₁ ~ ρ₁ | | |
| 3: | for s in [0,1/Δt) do | | |
| 4: | \| | t ← 1 − sΔt | |
| 5: | \| | Sample z ~ [symbol] (0,1) | |
| 6: | \| | dBₜ ← ζγ [symbol] · \sqrt{dt} · z | |
| 7: | \| | d\dot{B}ₜ ← hat(dBₜ) | [symbol] map rotation vector to so(3) |
| 8: | \| \| | ${u}_{t} ← {r}_{t}^{T} {v}_{θ} ( t , {r}_{t} )$ | [symbol] parallel-transport the vector field to so(3) |

| | | |
|---|---|---|
| 9: | └ | r_{t+Δt} ← r [symbol] ← exp( [symbol] + dBₜ [symbol] ) |

| | |
|---|---|
| 10: | return r₀ |

[symbol]

indicates data missing or illegible when filed

### Protein Task Hyperparameters

FOLDFLOW is implemented in pytorch, and uses the invariant point attention (IPA) implementations from OpenFold (Ahdritz et al., 2022) in the backbone. The Adam optimizer was used with constant learning rate 10⁻⁴, β₁=0.9, β₂=0.99. The batch size depends on the length of the protein to maintain roughly constant memory usage. In practice, set the effective batch size to

$$eff\_bs = max ( \# GPUs \times 500 , 000 / {N}^{2} ) , 1 ) \qquad ( 46 )$$

for each step. Set λₐᵤₓ=0.25 and weight the rotation loss with coefficient 0.5 as compared to the translation loss which has weight 1.0. Instead of the L2 loss on the rotation vector, separate the loss into two components: one on the axis and one on the angle for the rotation vector. This reduced variance and numerical instability in the training.

### Data and Data Sampling

A subset of PDB filtered with the same criteria as FrameDiff was used. Specifically, the following filter was applied: monomers of length between 60 and 512 (inclusive) with resolution <5A downloaded from PDB (Berman et al., 2000) on Jul. 20, 2023. After filtering out any proteins with >50% loops, 22248 proteins remain. To support diversity, sample uniformly over clusters with similarity of 30% as suggested in FrameDiff-Improved model. FOLDFLOW models function most efficiently with batches of proteins of the same length, so each batch contains proteins of a single length. There are 4268 clusters in the dataset.

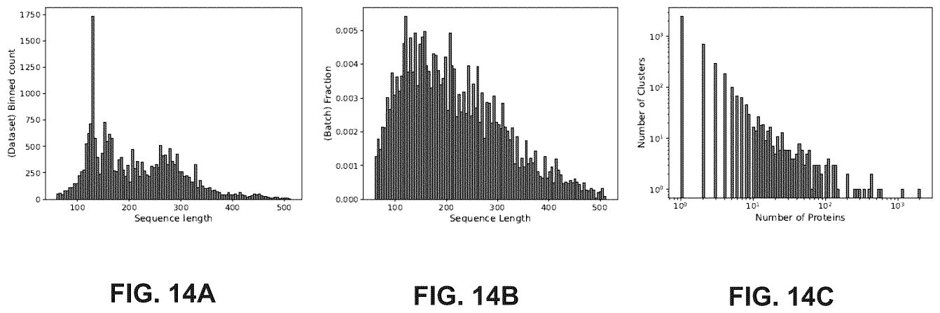

To assess the effects of the sampling methods on protein diversity and length distribution, FIGS. 14A-14C are shown. Specifically FIG. 14A illustrates the variability and range of protein lengths in the dataset, giving an overview of available lengths for sampling. FIG. 14B shows the batch fraction per length, highlighting alterations in sequence length distribution during training due to uniform cluster sampling. FIG. 14C which uses a log scale on both axes, unveils the variation in cluster sizes and the skewness in protein distribution across clusters. Uniform cluster sampling enhances batch diversity, aiding model generalization over various protein sequences and structures. However, as observed in FIG. 14B, it slightly modifies the sequence length distribution during training. FIG. 14C reveals an unevenness in protein distribution across clusters, with two bins containing approximately 14% of proteins.

### Protein Metrics

The TM-score. The template modeling score (TM-score) measures the similarity between two protein structures. The TM score can be expressed for two protein backbones X₀, X₁ ∈SE(3)^{N} as

$$TM - score ( {x}_{0} , {x}_{1} ) = max [ \frac{1}{{N}_{target}} {\sum }_{i}^{{N}_{c o m m o n}} \frac{1}{1 + {( \frac{{d}_{i}}{{d}_{0} ( {N}_{target )}} )}^{2}} ] \qquad ( 47 )$$

where N_{target} is the length of the target sequence, N_{common} is the length of the common sequence after 3D structural alignment, dᵢ is the distance (post alignment) of the i^{th} residues in x₀ and x₁, and

$${d}_{0} ( N ) = 1.24 {( N - 15 )}^{\frac{1}{3}} - 1.8$$

is a scaling factor to normalize across protein lengths. The TM-score ranges between (0,1] with a TM-score of 1 indicating perfectly aligned structure. In general a TM-score>0.5 are considered roughly similar folds, with TM-score <0.2 corresponding to randomly chosen unrelated proteins.

The RMSD metric. The root-mean-square deviation (RMSD) is a simple metric over paired residues expressed as

$$RMSD ( {x}_{0} , {x}_{1} ) = \sqrt{{\sum }_{i = 1}^{L} \frac{{d}_{1}^{2}}{L}} \qquad ( 48 )$$

where dᵢ is again the distance between the i^{th} residues heavy atoms [N, C_{α}, C, O]. The RMSD score is length dependent unlike the TM-score, but has been show to be a more stringent filtering step then TM-score >0.5 for designability (Watson et al., 2023). In general, as compared to TM-score the RMSD metric is more sensitive local errors and less sensitive to global misalignments.

Designability. A generated protein structure is considered designable if there exists an amino acid sequence which refolds to that structure. First, 50 proteins at lengths {100, 150, 200, 250, 300}, were generated then apply ProteinMPNN with sampling_temp=0.1 8 times to generate 8 sequences for every generated structure. Finally default ESMFold was applied and aligned RMSD of the Cα backbone atoms to calculate alignment of each ESMFold-refolded structure with the generated structure. A protein was deemed designable if at least one of the 8 refolded structures has an scRMSD score <2.0. While a threshold of <2.0 for designability is standard, this threshold may be unreasonably strict for longer backbones. However, it is unclear how this threshold should decay with increasing sequence length.

Finally, the imperfection of the self-consistency designability metric: when ESMFold does not produce the same structure as FOLDFLOW it does not imply FOLDFLOW's structure is wrong, especially for longer sequences where protein folding models are known to perform worse. Both ProteinMPNN and ESMFold are imperfect, and the failure cases of these models has not been well characterized. While, the false positive rate of this metric appears to be low, the false negative of this metric has not been quantified.

Diversity. All pairwise TM-scores were calculated for all generated structures that achieve the designability threshold of scRMSD <2 for each length of protein. The mean overall of these pairwise TM-scores was computed as the diversity metric. For this metric, a lower score is better. Diversity was compared on designable proteins to avoid inflation of designability scores by models which produce poor, proteins that may be very dissimilar to the space of refoldable proteins at that length.

Novelty. Novelty was calculated as the minimum TM-score of designable generated proteins to the training data. FOLDFLOW and FrameDiff use the most similar training datasets (only differing in about 10% of structures) where Genie and RF diffusion use substantially larger datasets. This may cause novelty to be overestimated for these models as there may be structures in their training sets that are far from the used training set.

### Conclusion

In conclusion, described herein is FOLDFLOW, a family of simulation-free generative models under the flow matching framework. Within this model class, FOLDFLOW-BASE learns deterministic dynamics over SE(3) and FOLDFLOW-OT learns more stable flows using Riemannian OT. To learn stochastic dynamics, FOLDFLOW-SFM learns an SDE over SE(3)^{N}, and was motivated by learning Brownian bridges over SO(3), but in a simulation-free manner. The empirical caliber of FOLDFLOW models was investigated on PDBs that contain up to 300 amino acids and the proposed models were competitive with RFDiffusion while significantly outperforming the current non-pretrained SOTA approach, FrameDiff-Improved, on all metrics. Finally, FOLDFLOW is more amenable for equilibrium conformation sampling which is an important subtask in protein design.

### Example 2: Example Machine Learning Models (e.g., FOLDFLOW++ Models) Further Outperform Conventional Methods in Generating Protein Backbone Structures

Disclosed herein is FOLDFLOW++, a novel protein structure generative model that is conditioned on protein sequences. FOLDFLOW++ was built on the foundations of FOLDFLOW and is an SE(3)^{N}-invariant generative model for protein backbone generation that handles multi-modal data by design. Specifically, FOLDFLOW++ introduces several new architectural components over previous protein structure generative models that enable it to process both 3D structure and discrete sequences. These include (1) a joint structure and sequence encoder; (2) a multi-modal fusion trunk that combines the representations from each modality in a shared representation space; and (3) a transformer-based geometric decoder. In contrast to prior efforts to incorporate sequences in structure-based generative models, FOLDFLOW++ leverages the representational power of a large pre-trained protein language model in ESM enabling it to make use of the rich biological inductive bias found in sequences but at a scale far beyond ground-truth experimental 3D structures found in the Protein Data Bank (PDB).

As a sequence-conditioned model, FOLDFLOW++ is able to tackle a suite of new tasks beyond simple unconditional generation. Specifically, the FOLDFLOW++ model can additionally be used for protein folding by simply generating structures conditioned on sequence as well as hard, biologically motivated conditional design problems. For instance, FOLDFLOW++ model can perform partial structure generation by conditioning on a masked sequence, i.e., structure in-painting. This enables FOLDFLOW++ to be better equipped than prior structure-only generative models to tackle the key challenges in de novo drug design. For example, in settings where the aim is to engineer a structure that binds and neutralizes a desired target protein structure and sequence pair; this is precisely a structure and sequence in-painting problem.

As diversity and quantity of training samples play a crucial role in downstream generative modeling performance on conditional design tasks, we construct a new large dataset—an order of magnitude larger than PDB—of high-quality synthetic structures filtered from SwissProt [Jumper et al., 2021, Varadi et al., 2021]. Further investigation was undertaken to determine the impact of fine-tuning FOLDFLOW++ using Reinforced Fine-Tuning (ReFT), a new approach that aligns flow-matching generative models to arbitrary rewards.

In the context of protein backbone generation, fine-tuning was applied to improve the properties of generated backbones, such as optimizing for the diversity of secondary structures, as well as improving the performance on conditional generation tasks like generating scaffolds around a target motif.

Main results. The main empirical results obtained using FOLDFLOW++ are summarized below:
FOLDFLOW++ achieved state-of-the art performance for unconditional generation and leads to the most designable, novel, and diverse proteins. In particular, FOLDFLOW++ improves over RFDiffusion [Watson et al., 2023] and FoldFlow [Bose et al., 2023].
FOLDFLOW++ closed the gap in performance with purpose-built folding models like ESMFold and improved by a factor of ˜ 4x compared to MultiFlow [Campbell et al., 2024], the most comparable protein structure generative model that also leverages sequences.
FOLDFLOW++ solved a biologically relevant conditional design problem in motif scaffolding. A fine-tuned FOLDFLOW++ was able to solve all 24/24 scaffolds in the benchmark dataset from Watson et al. [2023]. On challenging VHH nanobodies, it solved 9/25 refoldable motifs in comparison to 5/25 for the previous best approach RFDiffusion.
As ahypothesis, FOLDFLOW++ is able to perform zero-shot equilibrium conformation sampling on unseen proteins in the ATLAS molecular dynamics (MD) dataset [Vander Meersche et al., 2024] based on conformation variation seen within the protein data bank.
FOLDFLOW++ was able to capture different modes of the equilibrium conformation comparably to ESMFlow-MD [Jing et al., 2024], a model fine-tuned on MD data.

### Background and Preliminaries

### Protein Backbone and Sequence Parametrizations

Sequence representation. Protein sequences correspond to the chain of amino acids, which for a protein of length N is identified by a discrete token a^{l} ∈{1, . . . , 20}=: A. These discrete tokens were encoded using a one-hot representation. The entire amino acid sequence associated with a protein was denoted as A∈R^{N×20}.

Structure representation. The 3D structure of protein backbones can be represented as rigid frames associated with each residue in an amino acid sequence. Each residue, i, within a protein backbone of length N consists of idealized coordinates of their 4 heavy atoms N*, C*, C*, O*E R³, with C*_{α}=(0, 0, 0). The defining property of rigid frames is that they can be viewed as elements of the special Euclidean group SE(3) and as such each frame X=(r, s)∈SE(3) contains a rotation r and translation s component. Applying a rigid transformation x₁ to the idealized coordinates of the heavy atoms enables representation of the rigid frame of a given residue, [N, C_{α}, C, O]ⁱ=xⁱo[N*, C*_{α}, C*, O*], where o is the binary operator associated to the group, which for SE(3) is simply matrix multiplication. This leads to a structure representation of the complete 3D coordinates associated with all heavy atoms of a protein as the tensor X∈R^{N×4×3}.

SE(3): the group of rigid motions. The special Euclidean group SE(3) contains rotations and translations in three dimensions and can be thought of in several ways. It is a Lie group, i.e., a differentiable manifold endowed with a group structure. SE(3) can be seen as the group of rigid frames, representing 3D rotations and translations. As a Lie group, SE(3) can be uniquely identified with its Lie algebra, the tangent space at the identity element of the group. SE(3) is also a matrix Lie group, meaning that its elements can be represented with matrices. It can formally be written as the semidirect product of the rotation and the translation groups, SE(3)˜=SO(3) [symbol] (R3, +).

Flow matching on the SE(3) group

As Lie groups are smooth manifolds, they can also be equipped with a Riemannian metric, which can be used to define distances and geodesics on the manifold. On SE(3), a natural choice of the metric decomposes into the metrics on its constituent subgroups, SO(3) and R³. This allows building of independent flows on the group of rotations and translations and induce a flow directly on SE(3).

Probability paths on SO(3). Given two densities β₀, ρ₁ ∈SO(3), a probability path ρₜ: [0, 1]→P(SO(3)) is an interpolation, parametrized by time, t, between the two densities in probability space. Without loss of generality, consider ρ₀ to be the target data distribution and ρ₁ an easy-to-sample source distribution. A flow is a one-parameter diffeomorphism in t, ψₜ: SO(3)→SO(3). It is the solution to the ordinary differential equation (ODE).

$$\frac{d}{dt} ψ t ( r ) = ut ( ψ t ( r ) ) ,$$

with initial conditions ψ₀(r)=r, where uₜ is the time-dependent smooth vector ut: [0, 1] ×SO(3)→SO(3). It is said that ψt generates ρₜ if it induces a pushforward map ρₜ=[ψt]#(β₀).

Matching vector fields on SO(3). The framework of Riemannian flow matching [Chen and Lipman, 2023] can also accommodate Lie groups such as SO(3). Consequently, to learn a continuous normalizing flow (CNF) that pushes forward samples r₀˜ρ₀ to r₁˜ρ₁ regress a parametric vector field ν_{θ}∈X(SO(3)) in the tangent space of the manifold to the target conditional vector field uₜ (rₜ|r₀, r₁), for all t∈[0, 1]. Conveniently, the target uₜ (rₜ|r₀, r₁) is the time derivative of a point rₜ along the shortest path between r₀ and r₁—i.e., the geodesic interpolant rₜ=expᵣ₀ (t logᵣ₀ (r₁)). Furthermore, for SO(3) the target conditional vector field admits a closed-form expression

$${u}_{t} ( {r}_{t} \vert {r}_{0} , {r}_{1} ) = {log}_{{r}_{t}} \frac{{r}_{0}}{t}$$

as the experimental and logarithmic maps are numerically computable using the axis-angle representation of the group elements.

Given these ingredients, formulate the flow matching objective for SO(3) as:

$${L}_{SO ( 3 )} ( θ ) = {E}_{t ∼ U ( 0 , 1 ) , q ( {r}_{0} , {r}_{1} ) , {ρ}_{t} ( {r}_{t} \vert {r}_{0} , {r}_{1} )} {‖ {v}_{θ} ( t , {r}_{t} ) - {log}_{{r}_{t}} ( {r}_{0} ) / t ‖}_{SO ( 3 )}^{2}$$

In this equation, q(r₀, r₁) is any coupling between samples from the source and target distributions. An optimal choice is to set q(r₀, r₁)=π(r₀, r₁) which is the coupling, π, that solves the Riemannian optimal transport problem using minibatches [Bose et al., 2023, Tong et al., 2023, Fatras et al., 2020]. Finally, the generation of samples is done by first drawing from a source sample r₁˜ρ₁ and integrating the ODE backward in time using the learned vectorfield ∇_{θ}.

FOLDFLOW++ operates on protein backbones x₀˜ρ₀ which are parametrized as N rigid frames as well as their corresponding sequence a. As protein backbones contain symmetries, FOLDFLOW++ was designed as an SE(3)^{N}-invariant density using a flow-matching objective. Translation invariance was achieved by constructing the flow on the subspace SE(3)^{N}, where the center of mass of the inputs is removed. Additionally, focus was on building flows on the group of rotations SO(3) and translations R³, for each of the N residues independently as SE(3)^{N} can be viewed as a product manifold consisting of N copies of SE(3). The overall loss function for the model decomposes into per residue rotation and translation losses L=Lₛₒ₍₃₎+L_{R3},

$$L = {E}_{t ∼ U ( 0 , 1 ) , {ρ}_{t} ( {x}_{t} \vert {x}_{0} , {x}_{1} , \overline{a} )} [ {‖ {v}_{θ} ( t , {r}_{t} , \overline{a} ) - {log}_{{r}_{t}} ( {r}_{0} ) / t ‖}_{S O ( 3 )}^{2} + {‖ {v}_{θ} ( t , {s}_{t} , \overline{a} ) - \frac{{s}_{t} - {s}_{0}}{t} ‖}_{2}^{2}$$

where the pair (x₀, x₁)˜π(x₀, x₁) is sampled from the optimal transport plan π. In addition, the sequence a⁻=a (·) m corresponds to x₀ and is masked completely, with a mask m, with a probability Bern (0.5). Operationally, this means 50% of the time the model is trained unconditionally with no sequence information, i.e., a⁻=[Ø]^{N}, while the other 50% the model has access to the full sequence a⁻=a. Optimizing the loss is equivalent to maximizing the conditional log-likelihood of observing protein structures given their sequences log p (X|A) when the sequence is not masked and maximizing the unconditional log-likelihood log p (X) when the sequence is fully masked. Due to the ability to mask sequences, FOLDFLOW++ enables new modeling capabilities in comparison to existing models. More precisely, FOLDFLOW++ trained using masked sequences can perform a diverse set of tasks beyond simple unconditional backbone generation which aids in tackling more biologically relevant problems that require conditional generation such as mimicking a protein folding model and designing the 3D scaffolds around a target motif.

With the breadth of tasks T1-T3 (table 4) FOLDFLOW++ unlocks new structural design capabilities beyond the simple unconditional generation ability of FOLDFLOW.

TABLE 4
By manipulating the input modalities, FOLD- FLOW++ is able
to perform a diverse set of conditional and unconditional generation
tasks including biologically relevant tasks such as designing scaffolds.

| | Task Name | Sequence Inputs | Structure Inputs |
|---|---|---|---|
| (T1) | Unconditional | Fully-masked | Noise |
| (T2) | Folding | Unmasked | Noise |
| (T3) | In-Painting | Partially Masked | Partially Masked |

### Foldflow++

### FOLDFLOW++ Architecture

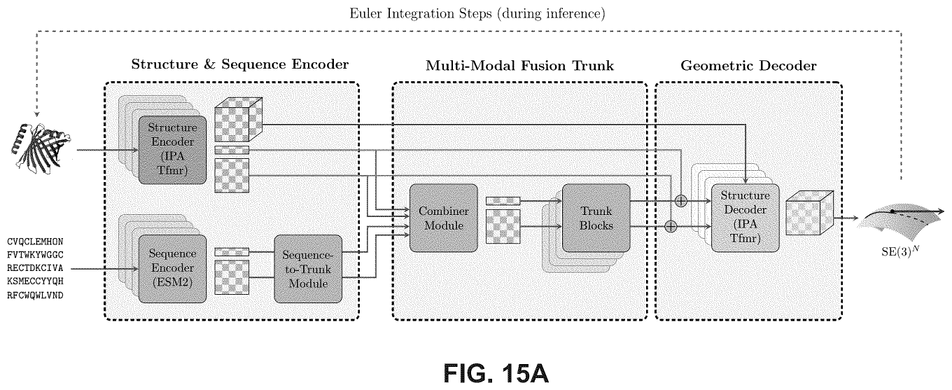

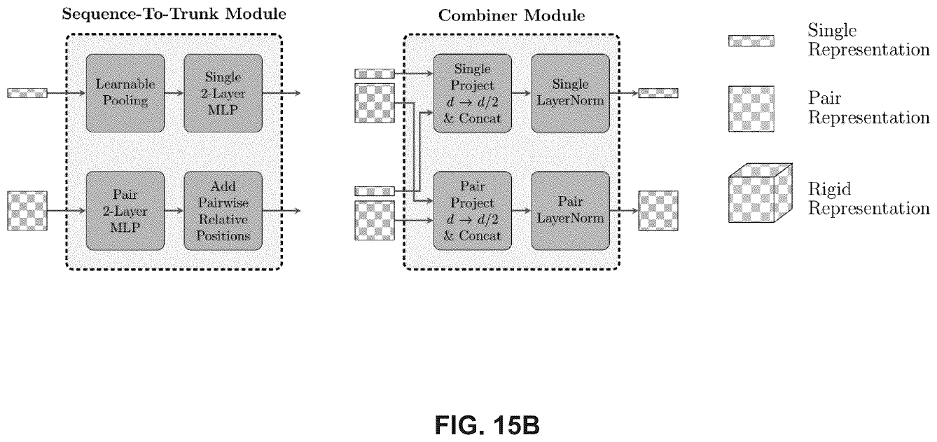

The FOLDFLOW++ architecture is comprised of three core components: (1) Structure and sequence encoder: An encoder which encodes both structures and sequences; (2) Multi-modal fusion trunk: the trunk which combines the multi-modal representations of the encoded structure and sequences; and (3) Geometric Decoder: a decoder that consumes the fused representation from the trunk and outputs a generated structure. The overall architecture of FOLDFLOW++ is depicted in FIGS. 15A-15B.

Structure and Sequence Encoder. Leverage existing state-of-the-art architectures to encode the structure and sequence modalities separately. For structure encoding, rely on the invariant point attention (IPA) transformer architecture, which is SE(3)-equivariant. The benefit of the IPA architecture is that it is highly flexible and can both consume and produce a structure—i.e., N rigid frames—and also output single and pair representations of the input structure. To encode amino-acid sequences, use a large pre-trained protein language model: the 650M variant of the ESM2 sequence model. Large protein language models have a strong inductive bias on atomic-level predictions of protein structures while exhibiting strong generalization properties beyond any known experimental structures—which we argue is highly correlated with goals of de novo structure design. Moreover, the ESM2 architecture also produces single and pair representations for an encoded sequence of amino acids, which conceptually correspond to the single and pair representations from the structure encoder. Consequently, the output space of each modality prescribes a natural fusion of representations into a joint single and pair latent space for a given input protein.

Multi-Modal fusion trunk. After encoding both input structure and sequence, a joint representation was constructed for the single and pair representation using a “project and concatenate” combiner module with simple MLPs. LayerNorm was used throughout the architecture as it accommodates differently-scaled inputs. The joint representations were further processed by a series of Folding blocks, which refines the single and pair representations via triangular self-attention updates.

Geometric decoder. To decode the joint representations of the inputs into SE(3) vector fields, the IPA Transformer architecture was leveraged. The decoder takes as input the single, pair outputs of the trunk and the rigid representations from the structure encoder. One of the major findings is that including a skip-connection between the structure encoder and the decoder contributes towards good performance as the temporal information is given to the structure encoder. Given each component, 2-2-2 blocks were stacked for the encoder, trunk, and decoder components.

### Training

FOLDFLOW++ was trained by alternating between both folding and unconditional generation tasks using a novel sequence-and-structure flow matching procedure, described below.

Dataset construction. The generalization ability of generative models trained using maximum likelihood is determined by the quality and diversity of curated training data. Due to the limited size of ground truth structures in the Protein Data Bank (PDB), the training set diversity was improved by additionally curating a dataset of filtered AlphaFold2 structures from Swis-sProt. To ensure FOLDFLOW++ was trained on high-quality synthetic structures, a set of stringent filtering techniques was employed that remove many undesignable proteins from SwissProt. After filtering, the final dataset consisted of 160K structures and constituted approximately an 8× fold increase compared to prior works [Yim et al., 2023b, Bose et al., 2023]. The exact layered filtering strategy for synthetic structures in SwissProt is outlined by the following steps:
(Step 1) Filtering low-confidence structures. Use per-residue local confidence metrics like the average pLDDT to filter out low-confidence structures from the initial SwissProt dataset.
(Step 2) Masking low-confidence residues. Globally high-confident structures may include low-confidence residues with disordered regions that can impede training. A per-residue pLDDT threshold of 70 was used to mask such “low-quality” residues during training.
(Step 3) Filter high-confidence, low-quality structures. The nature of synthetic data means that even following steps 1 and 2 low-quality data persists in a curated dataset. To combat this, further filter structures by learning a light-weight structure prediction model trained on structural features predictive of protein quality.

A detailed analysis of each step is described in the section entitled “Dataset filtering”, which includes examples of low-quality structures that were filtered as illustrative examples. During training, the fraction of synthetic samples that may be seen during an epoch was set to ⅔ of the epoch. This prevented the model from overfitting to the remaining noise in the synthetic data, and is also common practice when training with synthetic data. Anecdotally, no improvement was noticed from using a smaller proportion of synthetic structures. Finally, in the FOLDFLOW++ architecture, the ESM pre-trained language model was fixed during training and all other components (encoder, trunk, and decoder) were trained from scratch. The results use PDB data only, as this displayed the best performance for designability scores.

### Fine-Tuning FOLDFLOW++

The efficacy of fine-tuning FOLDFLOW++ was explored with preferential alignment. A supervised fine-tuning approach was undertaken, the supervised fine-tuning approach using an additional fine-tuning dataset which is filtered using pre-specified auxiliary rewards rₐᵤₓ to create a preferential dataset D_{pref}. This is termed “Reinforced FineTuning (ReFT)” since fine-tuning in this manner can be considered aligning FOLDFLOW++ generations to the auxiliary reward. Summarizing this in three steps: (1) Take a curated dataset of proteins with desirable metrics; (2) Use rₐᵤₓ to score the samples from step 1 and filter them to get a subset of high-scoring samples; (3) Improve FOLDFLOW++ by SFT on the filtered subset. Finetuning with ReFT optimizes the following optimization objective L_{REFT} (θ),

$$\frac{max}{{p}_{θ}} {L}_{REFT} ( θ ) {E}_{( x , a ) - Dpref} [ {r}_{aux} ( x ) log p θ ( x \vert a ) ] \qquad ( 3 )$$

Compared to recent alignment methods based on reward models, ReFT uses a filtered structure dataset to fine-tune FOLDFLOW++. Standard RL approaches seek to fine-tune generative model-based model-generated data and assume access to evaluating the reward function. This approach of maximizing L_{REFT} (θ) constructgs D_{pref} with auxiliary reward rₐᵤₓ, demonstrated by the improvement in secondary structure diversity.

### Experiments

FOLDFLOW++ was evaluated on multiple protein design tasks including unconditional generation, motif scaffolding, folding, fine-tuning to improve secondary structure diversity, and equilibrium conformation sampling from molecular dynamics trajectories.

Baselines. As main baselines for the unconditional generation task, FoldFlow++ was compared to pre-trained versions of FrameDiff [Yim et al., 2023b], Chroma [Ingraham et al., 2023], Genie [Lin and AlQuraishi, 2023], MultiFlow [Campbell et al., 2024], and RFDiffusion which is the current gold standard [Watson et al., 2023]. In conditional generation tasks like motif scaffolding, FoldFlow++ was compared against a conditional variant of FrameFlow [Yim et al., 2023a] as well as RFDiffusion. For protein folding, FoldFlow++ was compared against ESMFold [Lin et al., 2022] and MultiFlow which also leverages sequence information. Lastly, for conformational sampling the principal baselines are ESMFlow and AlphaFlow [Jing et al., 2024].

### Unconditional Protein Backbone Generation

Unconditional structure generation was evaluated using metrics that assess the designability, novelty, and diversity of generated structures. For each method, 50 proteins were generated, illustrated for FoldFlow++ in FIG. 16, at lengths {100, 150, 200, 250, 300}. Designability is computed by using the self-consistency metric which compared the refolded proteins (with ProteinMPNN [Dauparas et al., 2022] and ESMFold [Lin et al., 2022]) with the original one. Novelty was computed using: 1.) the fraction of designable proteins with TM-score <0.3 and 2.) the average maximum TM-score of designable generated proteins to the training data. Finally, for diversity, the average pairwise TM-score of the designable generated samples averaged across lengths was used as well as the maximum number of clusters.

Results. FoldFlow++ outperformed all other methods on all metrics crucially without requiring a pretrained folding model as part of the architecture like RFDiffusion. In particular, FoldFlow++ produced the most designable samples with 97.6% of samples being refolded by ESMFold to within <2 Å. FoldFlow++ novelty improved over RFDiffusion by an absolute 25.2% in the fraction of designable samples with TM-score <0.3. Furthermore, a 19.9% and 102.3% relative improvement was observed in the diversity of FoldFlow++ over RFDiffusion as measured by the pairwise TM-score and Max Cluster fraction. This places FoldFlow++ as the current most designable, novel, and diverse protein structure generative model.

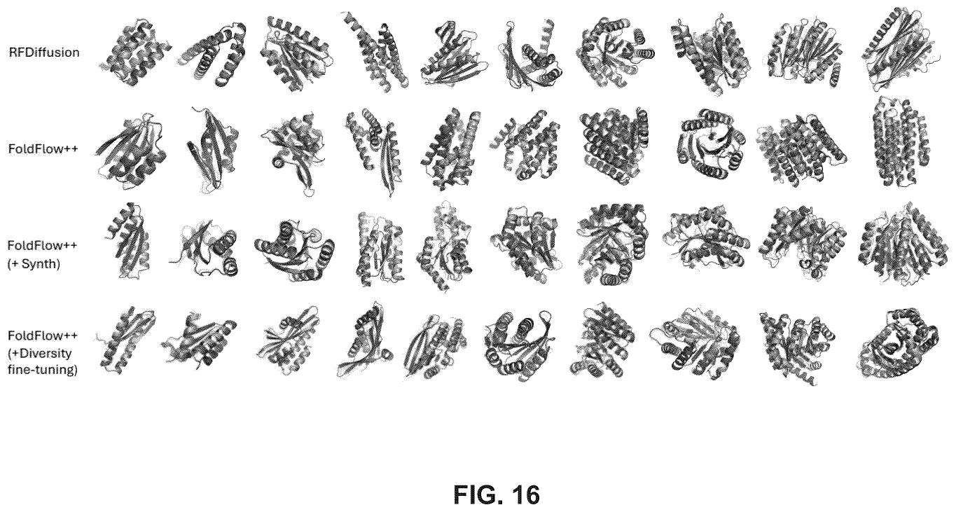

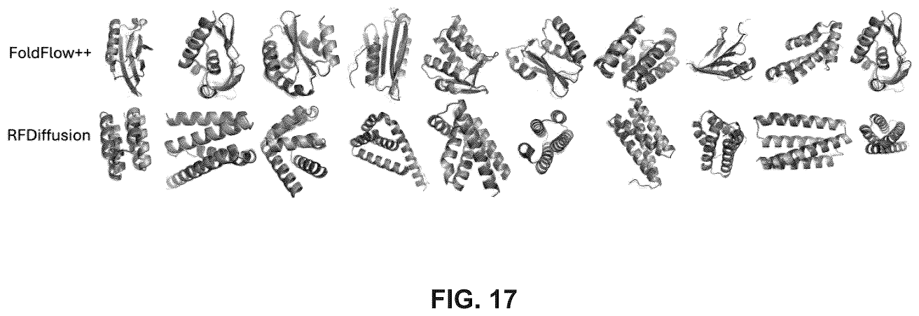

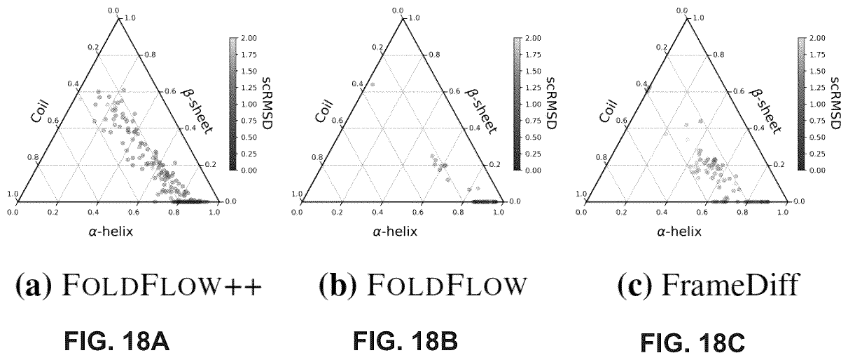

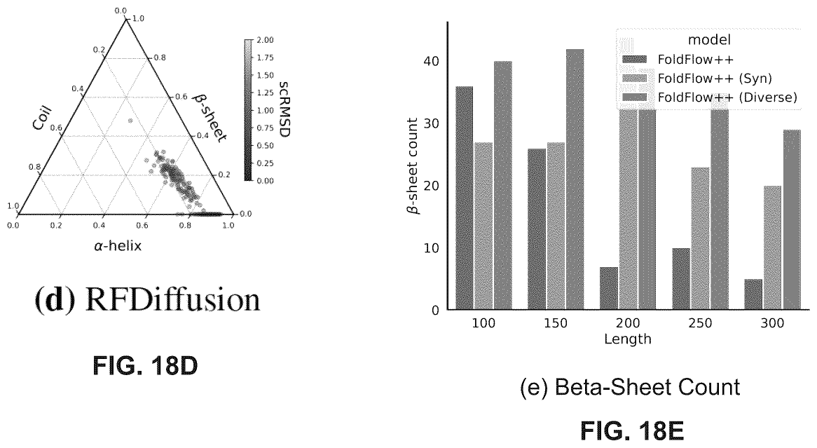

Further presented are uncurated generated samples of FOLDFLOW++ and RFDiffusion in FIG. 17. Furthermore, the distribution of secondary structures of all methods was visualized in FIGS. 18A-18D. A clear indication was observed that FOLDFLOW++ was able to produce the most diverse secondary structures-more closely matching the training distribution and improving over RFDiffusion. Increased amounts of 8-sheets and coils were further observed, which are particularly challenging for models like FrameDiff and FOLDFLOW that primarily generate α-helices. We also include multiple ablations on architectural choices, inference annealing, and sequence conditioning in Table 5.

TABLE 5
Ablation study on FOLDFLOW++ (FF++) using: synthetic data, folding blocks, and stochastic
flow matching (SFM). We generated 250 proteins (50 of length 100, 150, 200, 250) and compared Designability
(fraction with scRMSD < 2.0 Å), Novelty (max. TM-score to PDB and fraction of proteins with
averaged max. TMscore < 0.3 and scRMSD < 2.0 Å), and Diversity (avg. pairwise TMscore and MaxCluster fraction).

| | | Novelty | | Diversity | | | | | | |
|---|---|---|---|---|---|---|---|---|---|---|
| | Designability | Frac. | Avg/max | p.wise | MaxClust. | Seq. | Folding | | | # train |
| | Frac. < 2 Å (↑) | TM < 0.3 (↑) | TM (↓) | TM (↓) | (↑) | cond. | blocks | SFM | iter/s | param. |
| FF++(−F. Block − | 0.716 ± 0.029 | 0.188 ± 0.025 | 0.419 ± 0.012 | 0.240 | 0.228 | X | X | X | 2.7 | 17M |
| ESM2) | | | | | | | | | | |
| FF++(−F. Block) | 0.852 ± 0.023 | 0.148 ± 0.023 | 0.438 ± 0.010 | 0.227 | 0.271 | ✓ | X | X | 2.1 | 18M |
| FF++ | 0.976 ± 0.010 | 0.368 ± 0.031 | 0.636 ± 0.009 | 0.206 | 0.348 | ✓ | ✓ | X | 1.6 | 21M |
| FF++(+Synthetic) | 0.785 ± 0.027 | 0.047 ± 0.014 | 0.465 ± 0.008 | 0.226 | 0.264 | ✓ | ✓ | X | 1.6 | 21M |
| FF++(+SFM) | 0.935 ± 0.016 | 0.274 ± 0.029 | 0.386 ± 0.009 | 0.218 | 0.281 | ✓ | ✓ | ✓ | 1.5 | 21M |

Increasing Secondary Structure Diversity with Finetuning

ReFT based data filtering was investigated to improve diversity of secondary structures in generated samples. We use a diversity score based auxiliary reward for filtering, based on weighted entropy on the proportions of each residue belonging to each type of secondary structure—i.e., alpha-helices (a), coils c, beta-sheets β in the set S, that can be analytically written as r_{diversity}=(Σ_{s∈s} Pₛ log pₛ). Due to models producing increasing amounts of helices, use wₐ=1, W_{c}=0.5 and w_{β}=2, and take top 25% of samples according to the r_{diversity}. Experimental results in FIG. 18E with generated samples in FIG. 16 demonstrate that protein at all lengths benefit from training with ReFT as measured by diversity of generated samples, and produces most amount of β-sheets, and can surpass diversity improvement already obtained by training using synthetic structures as in FIG. 4.

### Folding Sequences

Given that FOLDFLOW++ is sequence conditioned, we can perform protein folding by providing a valid sequence during inference. During training, FOLDFLOW++ tries to transform a SE(3)^{N} noise sample into the given sequence's 3D structure. Therefore, we aim to measure the generalization properties of our model to fold unseen sequences. We evaluate folding on a test set of 268 unseen proteins from the PDB dataset. We compare the folding capabilities of FOLDFLOW++, ESMFold, and Multiflow. Table 6 reports the aligned RMSD between the predicted backbone and the ground truth backbone. FOLDFLOW++, trained for structure generation, approaches the performance of ESMFold which is a purpose-built folding model. Furthermore, FOLDFLOW++˜ 4× is better at folding than the most comparable model in MultiFlow [Campbell et al., 2024] which is a multi-modal flow matching model using sequences.

TABLE 6
Folding model evaluation on a test set of 268 proteins from PDB.

| Model | RMSD (↓) |
|---|---|
| ESMFold | 2.322 ± 4.270 |
| Multiflow | 14.995 ± 3.977 |
| FoldFlow++ | 3.237 ± 4.145 |

### Motif Scaffolding

In motif scaffolding, we are tasked with designing a subset of residues, termed “scaffolds”, around one or more subsections of a (“motif”′) protein structure that have known biologically-important functions through its interaction with a target. This enables the design of proteins with a priori functional sites using generative models. The motifs can be small and have non-specific shapes (e.g. a helix), and hence it is important for the generative model to understand the chemical information it carries on top of its geometry. The task of motif scaffolding is an example of how our model can be fine-tuned for conditional generation tasks. We consider two datasets for evaluating motif scaffolding performance: the benchmark proposed in Watson et al. consisting of 24 single-chain motifs, and a new benchmark based on scaffolding the Complimentary Determining Regions (CDRs) of VHH nanobodies, as found in the Structural Antibody Database [Schneider et al., 2022].

Motif Scaffolding Benchmark. We use the scaffolding benchmark from Watson et al. and follow the pseudo-label fine-tuning procedure described in Yim et al. [2023b] by randomly generating motifs from proteins by training on both the motif structure and sequence. For inference, we sample the scaffold lengths for each motif and provide both the both partially masked structure and sequence to the model. We follow the same evaluation procedure used in RFDiffusion. The results in Table 7 show that both FOLDFLOW++ and RFDiffusion solve all 24/24 motifs.

CDR Scaffolding. VHH antibodies, also known as nanobodies, have shown significant promise in protein design and therapeutics due to their unique properties [Muyldermans, 2021]. They are composed of a single variable domain derived from camelid heavy-chain antibodies, featuring three complementarity-determining regions (CDRs) that confer specificity and variability in antigen binding. As a result, creating effective scaffolds for nanobodies is challenging due to the need to maintain the designability of the CDRs and especially because any scaffolding effort must avoid altering these characteristics to preserve binding functionality. We treat this as a conditional generative modeling problem and fix the motif atoms, and mask the scaffold sequence information. Exact training and experimental details along with additional metrics are provided below. The results are found in Table 7, where the average motif scRMSD is much higher than the average scaffold scRMSD. The result is a much lower number of solved motif scaffolding.

TABLE 5
Motif-scaffolding benchmarks. FrameFlow does not have public code for
motif-scaffolding and thus cannot be evaluated on the VHH benchmark.

| | Benchmark | | | |
|---|---|---|---|---|
| | RFDiffusion Benchmark | VHH Benchmark | | |
| Model | Solved/24 | Motif | Scaffold | Solved/25 |
| RFDiffusion | 24 | 3.94 ± 1.54 | 2.40 ± 0.93 | 5 |
| FrameFlow + FT* | 21 | — | — | — |
| FOLDFLOW++ + FT | 24 | 2.78 ± 1.01 | 1.67 ± 0.24 | 9 |

“+FT” indicates “with fine-tuning”.
*Using reported numbers with AlphaFold2 instead of ESMFold used in our evaluation procedure.

### Zero-shot Equilibrium Conformation Sampling

We now test FOLDFLOW++ on zero-shot equilibrium conformation sampling task. Starting from a sequence, we generated multiple conformations of the same proteins and compared the distribution of conformations with the ones from molecular dynamic simulations. We compared FOLDFLOW++ with AlphaFlow-MD and ESMFlow-MD; two folding models fine-tuned on a molecular dynamic dataset.

Table 8 reports the pairwise and global RMSD, the root mean square fluctuation (RMSF), and the 2-Wasserstein on the top two principal components. For both RMSD and the RMSF metrics, we report the Pearson correlation between the values from the generated ensemble and those of the ground truth ensemble.

We use the same test set as in Jing et al. restricted to proteins of length at most 400 amino acids. Notably FOLDFLOW++ performs similarly or better than the comparable model ESMFlow-MD across all metrics without any fine-tuning and with significantly fewer parameters on molecular dynamics data, indicating that the base model trained only on PDB already captures similar information about protein dynamics as models given explicit access to this data. Moreover, FOLDFLOW++ requires 4.5× less GPU hours for training and 33× less trainable parameters while allowing for 6× faster inference steps than ESMFlow-MD as reported in Table 9, improving FOLDFLOW++'s prospects as a practical base model for future work on capturing protein dynamics.

TABLE 8
Zero-shot performance of the base FOLDFLOW++ model on the ATLAS dataset of MD trajectories
compared to ESMFlow and AlphaFlow models fine-tuned on ATLAS. FOLDFLOW++ is competitive
to the comparable model ESMFlow across all metrics. r denotes Pearson's correlation coefficient.

| Model | Pairwise RMSD r (↑) | Global RMSF r (↑) | Per-target RMSF r (↑) | PCA W₂-dist (↓) |
|---|---|---|---|---|
| AlphaFlow-MD | 0.468 ± 0.005 | 0.415 ± 0.006 | 0.824 ± 0.000 | 10.67 ± 0.29 |
| ESMFlow-MD | 0.293 ± 0.005 | 0.161 ± 0.005 | 0.737 ± 0.001 | 11.51 ± 0.13 |
| FOLDFLOW++ | 0.297 ± 0.004 | 0.236 ± 0.004 | 0.658 ± 0.001 | 10.85 ± 0.15 |

TABLE 9
Molecular dynamics experiment training details.

| Model | # training GPU hours | # total parameters | # trainable parameters | Inference time/step (sec) |
|---|---|---|---|---|
| AlphaFlow-MD | 2224 | 95M | 95M | 3.26 ± 0.01 |
| ESMFlow-MD | 872 | 3.5B | 694M | 1.12 ± 0.01 |
| FOLDFLOW++ | 192 | 672M | 21M | 0.18 ± 0.00 |

### Conclusion

Disclosed herein is a new sequence-conditioned protein structure generative model called FOLDFLOW++. FOLDFLOW++ leverages a protein language model to condition Flow Matching-based protein generative models with sequences. The model achieves state-of-the-art results on unconditional generation and generates diverse and novel proteins, especially when trained on the new dataset. Conditioning over sequences allows the model to perform novel tasks such as folding sequences and motif-scaffolding tasks and we show its competitiveness on those tasks.

### Additional Methods and Analysis

### Dataset Filtering-PDB Structures

We used a subset of PDB with resolution <5 Å downloaded from the PDB [Berman et al., 2000] on Jul. 20, 2023. Standard filtering was performed to remove any proteins with >50% loops. During preprocessing, we also removed any non-organic residues at either end of the structure. In previous works, these residues are typically kept but masked during training, however they contributed to the total forward pass FLOPs and therefore decrease training efficiency. By removing these residues during preprocessing, this increases the number of training examples per batch. Finally, the PDB dataset was re-clustered using mmseqs2 at the 50% sequence identity threshold to obtain 6,593 clusters during training.

### Synthetic Structures

Global pLDDT Filtering. We first filtered out globally low-confident structures, as measured by average and standard deviation of pLDDT taken across all residues in a predicted structure. Despite SwissProt already being a curated set of high-confidence structures, we still found considerable variation in quality. The final filtering criteria were (avg pLDDT >85) && (std pLDDT <15) to keep only “consistently good” structures.

High-Confidence Low-Quality Filtering. Despite the global pLDDT filtering, there were still low-quality structures in the training after global pLDDT filtering. Low-quality structures are characterized by having high overall confidence and good local qualities but unrealistic global structures or sub-chain interactions. These structures easily corrupt the training data and cause a model to produce similarly “unfolded” generations.

### Model Architecture Details

We provide details for the IPA blocks and Folding blocks. For the IPA blocks, we follow the setting developed in [Yim et al., 2023b] by adding to the original IPA [Jumper et al., 2021] a skip connection and transformer layer on the node representation. The IPA modules takes as inputs the single and pair representations and a structure. For the structure encoder, we initialize the single and pair representations with positional and time embedding passed to MLPs. In our experiment, we used a node embedding dimension of 256, an edge dimension of 128, and a hidden dimension of 256. These settings are the same for both the encoder and decoder. We have use a skip connection between the representations of the structure encoder and decoder. We combine the single and pair representations of different modalities by projecting each with a linear layer of output dimensions 128 for the single representations and 64 for the pair representation. We then concatenate all modalities' representation to obtain a single representation of dimension 128 and pair representation of dimension 256. The Folding blocks are composed of 2 Triangular Self-Attention Blocks with single and pair head width of size 32. Finally, the pair and single representation dimensions are of 128. The structure decoder's single and pair representations inputs are an average between the refined ones from the Folding blocks and the initial ones. All other architectural details not specified here are set to the defaults of IPA or ESM, respectively.

### Training Details

Hyperparameters. See Table 10 for an overview of the experimental setup. We train with the “length batching” scheme in which each batch consists of the same protein sampled at different times. The number of samples in a batch is variable and is approximately [num_residues²/M] where Mis a hyperparameter in Table 10.

Training hardware setup. FOLDFLOW++ is coded in PyTorch and was trained on 2 A100 40 GB NVIDIA GPUs for 4 days. Initial tests runs were trained in a similar setting.

TABLE 10
Overview of Training Setup

| Training Parameter | Value |
|---|---|
| Optimizer | ADAM Kingma |
| | and Ba [2014] |
| Learning rate | 0.0001 |
| β₁, β₂, ϵ | 0.9, 0.999, 1e−8 |
| Effective M (max squared residues per batch) | 500k |
| % of experimental structures per epoch | 33% |
| Minimum number of residues | 60 |
| Maximum number of residues | 384 |
| Sequence masking probability | 50 |

TABLE 11
An overview of training time.

| | | | Total |
|---|---|---|---|
| Model | # Steps | # GPUs | Time (days) |
| RFDiffusion* [Watson et al., 2023] | 25k | 64 + 8 | 28 + 3 |
| FOLDFLOW [Bose et al., 2023] | 600k | 4 | 2.5 |
| FOLDFLOW++ ** | 500k | 2 | 4 |
| FOLDFLOW++ ** w/o folding block | 500k | 2 | 2.5 |

*RFDiffusion initializes from RoseTTAFold, and we include that training time in the estimates.
** We recall that FOLDFLOW++ uses frozen ESM2-650M which was trained on 512 GPUs for 8 days.

### Inference Details

The inference was performed with Euler integration steps. In the experiments, 50 steps gave state-of-the-art results. We use the Inference Annealing trick from Bose et al. [2023] multiplying the rotation vector by 10t, where t∈[0, 1] is the time parameter. We tried different scaling parameters and as found in [Bose et al., 2023], 10t provided the best performance.

### Unconditional Protein Backbone Generation

Metrics. Several quantity of interest were computed to measure the performance of FOLDFLOW++.
i) Start with designability, measured using the self-consistency metric with ProteinMPNN [Dauparas et al., 2022] and ESMFold [Lin et al., 2022], counting the fraction of proteins that refold (C_{α}-RMSD (scRMSD)<2.0 Å) over 50 proteins at lengths {100, 150, 200, 250, 300}. We also provide the average self-consistency rmsd over all generated proteins.
ii) In order to use generative models for drug discovery applications, the difference and novelty of the generated data was compared to training data. Novelty was measured using two metrics: 1.) the fraction of generated proteins that are both designable and are dissimilar to PDB structures (quantified by template-match score to PDB, i.e., PDB-TM score)<0.3, as used in Lin and AlQuraishi [2023], higher is better) and 2.) for designable proteins, the average closest similarity to training data (quantified by the maximum TM score, lower is better). The threshold for similarity has been studied previously, where the average TM-score on random structure pairs is ˜ 0.3 [Zhang et al., 2006].
iii) Finally, we desire a model that generates diverse proteins and not just of the same type. Proteins are usually gathered into different clusters during training. So for diversity, we use the number of generated clusters with a TM-score threshold of 0.5 [Herbert and Sternberg, 2008] (higher is better) as well as the average pairwise TM-score of the designable generated samples averaged across lengths as our diversity metric (lower is better). Note that in certain model, designability is inversely correlated with diversity as these models can produce unrealistic (e.g. unfolded) proteins that are “diverse” because they do not align well with each other.

Evaluation procedure. We followed the same evaluation procedure as in Watson et al. [2023], Yim et al. [2023b]. In particular, to evaluate the designability of a scaffold, we use ProteinMPNN [Dauparas et al., 2022] to decode 8 sequences and then re-fold those sequences, fixing the motif sequence which is known a priori. Given these re-folded structures, we compare three numbers:
1. Global RMSD: the overall aligned RMSD between the entire generated structure and the refolded structure.
2. Motif RSMD: the RMSD of the refolded motif residues aligned to the original motif residues.
3. Scaffold RMSD: the RMSD of the refolded scaffold residues aligned to the generated scaffold residues.

Following Watson et al. [2023], a scaffold is considered “designable” if the Global RMSD is <2 AND the motif RMSD <1 AND the scaffold RMSD <2. A detailed breakdown of our results can be found in Table 12.

TABLE 12
A detailed breakdown of FOLDFLOW++ motif scaffolding performance using
ESMFold to refold all structures. All numbers are out of 100 samples.

| | FOLDFLOW++ | | | | |
|---|---|---|---|---|---|
| | # Overall | | # Scaffold | | RFDiffusion # |
| Example | Valid | # Motif Valid | Valid | # Designable | Designable |
| 1BCF | 98 | 98 | 98 | 98 | 100 |
| 2KL8 | 95 | 95 | 95 | 95 | 100 |
| 1PRW | 77 | 99 | 99 | 77 | 91 |
| 6EXZ_long | 70 | 80 | 82 | 68 | 91 |
| 6EXZ_med | 60 | 73 | 76 | 60 | 87 |
| 1QJG | 73 | 62 | 93 | 54 | 80 |
| 5TRV_long | 48 | 49 | 85 | 36 | 77 |
| 5TRV_med | 50 | 46 | 84 | 33 | 80 |
| 6E6R_long | 35 | 72 | 95 | 31 | 82 |
| 6EXZ_small | 30 | 41 | 49 | 30 | 28 |
| 6E6R_med | 28 | 84 | 95 | 27 | 87 |
| 4ZYP | 36 | 53 | 87 | 25 | 85 |
| 6E6R_small | 24 | 81 | 97 | 21 | 50 |
| 1YCR | 22 | 91 | 98 | 20 | 91 |
| 7MRX_small | 18 | 31 | 67 | 15 | 22 |
| 5TPN | 14 | 32 | 59 | 14 | 79 |
| 5TRV_small | 31 | 22 | 75 | 13 | 53 |
| 3IXT | 14 | 89 | 93 | 13 | 85 |
| 5YUI | 24 | 12 | 40 | 9 | 8 |
| 7MRX_med | 17 | 32 | 66 | 6 | 22 |
| 7MRX_long | 15 | 21 | 68 | 4 | 22 |
| 5IUS | 15 | 4 | 56 | 3 | 11 |
| 4JHW | 12 | 3 | 38 | 1 | 8 |
| 5WN9 | 1 | 6 | 9 | 1 | 1 |

The choice of folding model appears to have a nontrivial impact on this metric. In their original papers, Watson et al. [2023], Yim et al. [2023b] used AlphaFold2 with no MSA and 0 recycles to refold their structures; however ESMFold is known to be significantly more accurate when no MSAs are provided [Lin et al., 2023]. Given this, we generated new samples from RFDiffusion (FrameFlow doesn't have public code for generating scaffolds) and re-folded them with ESMFold. The result is that RFDiffusion is able to solve all 24 examples; an increase of 4 vs. their reported numbers. Moreover, the proportion of solved increases relative to their reported results, suggesting that the accuracy of the folding model significantly impacts the ability to measure scaffold quality in silico.

### Pseudo-Label Training

We follow a similar data augmentation procedure as in [Yim et al., 2024, Watson et al., 2023], with the only modification of adding a minimum number of contiguous residues per motif. We use the same min length and adding an absolute minimum length of 2 for a motif. We continue training FOLDFLOW++ for 330,000 steps on the same dataset, with a learning rate of 10e-5. This was done using 2 A100 80G, for 2.85 days.

### Vhh Cdr

From the Structural Antibody Database we used 615 nanobody sequences, yielding 1831 chains from the PDB as training set, and for testing we used 40 sequences appearing in 106 PDB chains.

With both the sequence and CDRs readily available, we can build the appropriate mask for training, which is used to fix the motif atoms, and mask the scaffold sequence information. For testing, we sampled the scaffold segment lengths based on the median value of each scaffold segment, ±5.

Empirically the scaffold segment lengths varied less than that amount; nanobodies are known to exhibit less variability across their framework regions, both in sequence and structure [Mitchell and Colwell, 2018]. We continue training FOLDFLOW++ using this dataset, training for 10,000 steps with a learning rate of 10e-5. Using a single A100 80G this process takes 3.5 hours. We observed rapid increase but rapid tapering in performance using the VHH dataset; this is likely due to the lack of variability of the scaffolds themselves.

We note the designability metric used here and in other papers shows certain limitations when applied to this dataset and this task. Using the test set sequence and structures themselves, we compute the same scRMSD scores, in order to benchmark what should be, in theory, the best possible performance.

We observe that out of the 106 testing chains, only 25 of them are “solved” according to Watson et al. [2023]'s criteria. This raises the question as to whether or not this particular criteria and setup is applicable to any motif scaffolding task. We provide the full set of results in Table 15 on all 106 chains, and on a subset of size 25 in Table 14. In addition, the particular number of solved samples are reported in Table 13.

TABLE 13
A detailed breakdown of FOLDFLOW++ motif scaffolding performance
applied to refoldable VHH structures. The same comments as table
12 apply to evaluation. All numbers are out of 25 samples.

| | FOLDFLOW++ | | | | |
|---|---|---|---|---|---|
| | # Overall | | # Scaffold | | RFDiffusion # |
| Example | Valid | # Motif Valid | Valid | # Designable | Designable |
| 6qtl-B | 4 | 21 | 16 | 4 | 0 |
| 6qtl-G | 4 | 19 | 18 | 4 | 0 |
| 6rpj-H | 4 | 21 | 21 | 4 | 0 |
| 6rpj-D | 3 | 23 | 19 | 3 | 0 |
| 6qtl-F | 2 | 22 | 18 | 2 | 0 |
| 7epb-C | 2 | 35 | 33 | 2 | 4 |
| 6rpj-F | 3 | 24 | 21 | 2 | 0 |
| 6qtl-C | 3 | 20 | 14 | 2 | 1 |
| 5l21-B | 1 | 16 | 7 | 1 | 1 |
| 6oz6-G | 0 | 16 | 2 | 0 | 0 |
| 6oz6-E | 0 | 17 | 1 | 0 | 0 |
| 6oyz-G | 0 | 12 | 0 | 0 | 0 |
| 6oyz-F | 0 | 15 | 0 | 0 | 0 |
| 6oyh-H | 0 | 17 | 0 | 0 | 0 |
| 6oyh-G | 0 | 19 | 1 | 0 | 0 |
| 6gs7-H | 1 | 19 | 9 | 0 | 2 |
| 1kxq-E | 0 | 23 | 13 | 0 | 0 |
| 6rpj-B | 0 | 21 | 19 | 0 | 0 |
| 7a50-C | 0 | 23 | 18 | 0 | 0 |
| 7epb-D | 0 | 22 | 19 | 0 | 2 |
| 7o31-X | 0 | 14 | 12 | 0 | 0 |
| 7q3q-B | 0 | 13 | 5 | 0 | 0 |
| 7tjc-B | 0 | 17 | 9 | 0 | 0 |
| 8cxr-E | 0 | 14 | 1 | 0 | 0 |
| 8cxr-F | 0 | 18 | 1 | 0 | 0 |

TABLE 14
VHH Motif Scaffolding results on the re-foldable examples only. Reported are
the global, motif, and scaffold RMSD along with the number of solved tasks.

| | Global scRMSD | Motif scRMSD | Scaffold scRMSD | Solved (out of 25) |
|---|---|---|---|---|
| RFDiffusion | 3.1 ± 1.23 | 3.94 ± 1.54 | 2.4 ± 0.93 | 5 |
| FOLDFLOW++ VHH | 2.27 ± 0.5 | 2.78 ± 1.01 | 1.67 ± 0.24 | 9 |
| Test Set | 0.55 ± 0.19 | 0.65 ± 0.18 | 0.48 ± 0.19 | 25 |

TABLE 15
VHH Motif Scaffolding results on all samples, same numbers as Table 14

| | | | Scaffold | |
|---|---|---|---|---|
| | Global scRMSD | Motif scRMSD | scRMSD | Solved (out of 106) |
| RFDiffusion | 2.86 ± 1.1 | 3.6 ± 1.42 | 2.25 ± 0.85 | 18 |
| FOLDFLOW++ VHH | 2.3 ± 0.47 | 2.99 ± 0.86 | 1.66 ± 0.31 | 15 |
| Test Set | 1.49 ± 1.38 | 2.17 ± 1.22 | 1.05 ± 1.69 | 25 |

### Zero-Shot Molecular Dynamics

To evaluate FOLDFLOW++'s ability to capture protein dynamics we evaluated its performance on the test set of ATLAS [Vander Meersche et al., 2023] molecular dynamics used by Jing et al. but restricted to proteins at most 40 amino acids in length. To measure performance we used the following metrics. All metrics were computed exclusively over backbone atoms.
Pairwise RMSD r. To measure FOLDFLOW++'s ability to capture protein flexibility we first compute the average pairwise RMSD between every pair of conformations generated by each method. We then evaluate the average pairwise RMSD for the ground truth data and report the Pearson correlation r between the average pairwise RMSD per generated protein and ground truth data.
Global and per-target RMSF r. To further investigate flexibility, we measure the RMSF both globally and per target and compute the Pearson correlation r to ground truth data. Global RMSF is computed by, for each target calculating the backbone RMSF and taking its average, then measuring Pearson correlation between generated and ground truth samples over the sequence of averaged RMSFs. For per-target RMSF we instead compute the Pearson correlation between generated and ground truth backbone atom RMSFs and report the average taken over all targets.
PCA W₂. Following Jing et al. we seek to measure the distributional accuracy of our generated samples by evaluating the 2-Wasserstein distance using the first two principal components given by ground truth data. We use a PCA as evaluating W₂ on all atom coordinates would yield inaccurate measurements due to W₂ needing samples exponential in dimensionality in order to obtain reasonable estimates. We compute the PCA W₂ by, for each target, computing the first two principal components of backbone atom coordinates from ground truth MD data and projecting the backbone coordinates of each method's generated samples onto these coordinates. We then compute the W₂ per target and report the averaged W₂ over all targets.

As the test set contains 30,000 frames per protein computing test metrics using all ground truth conformations would be computationally infeasible. As such, following Jing et al. [2024] we randomly sample 300 conformations for each protein to be used as the test set. Samples were generated from FOLDFLOW++ using 50 inference steps and the inference annealing trick wherein the rotation vector is multiplied by 10t.

Table 16 details on the resources required for training FOLDFLOW++ compared to AlphaFlow-MD and ESMFlow-MD. FOLDFLOW++ requires an order of magnitude less time per inference step than ESMFlow-MD and AlphaFlow-MD while attaining results competitive with ESMFlow-MD while using 4.5× less GPU hours for training and 33× less trainable parameters.

FOLDFLOW++, ESMFlow-MD, and AlphaFlow-MD were all done on NVIDIA A100s. Inference time benchmarks were done on an NVIDIA A100, performing inference on a single protein of length 300 amino acids.

TABLE 16
Molecular dynamics experiment training details.

| Model | # training GPU hours | # total parameters | # trainable parameters | Inference time/step (sec) |
|---|---|---|---|---|
| AlphaFlow-MD | 2224 | 95M | 95M | 3.26 ± 0.01 |
| ESMFlow-MD | 872 | 3.5B | 694M | 1.12 ± 0.01 |
| FOLDFLOW++ | 192 | 672M | 21M | 0.18 ± 0.00 |

### Ablation Study and Sensitivity Analysis

Provided in this section are several ablations of the FOLDFLOW++ method. Table 5 provided the unconditional generation performance for the different architecture components of our FOLDFLOW++ method. Tables 17 and 18 compare the performance achieved by our model FOLDFLOW++ when we use different inference annealing t values for respectively unconditional generation and folding. In Tables 19 and 20, we compared the performance achieved by our model FOLDFLOW++ when using different numbers of Euler steps at inference for respectively unconditional generation and folding.

TABLE 17
Comparison of Designability (fraction with scRMSD < 2.0 Å), Novelty
(max. TM-score to PDB and fraction of proteins with averaged max. TMscore
< 0.3 and scRMSD < 2.0 Å), and Diversity (avg. pairwise TMscore
and MaxCluster fraction) for different inference annealing t values.

| | Designability | | | | |
|---|---|---|---|---|---|
| | Frac. < 2 Å | Novelty | | Diversity | |
| | (↑) | Frac. TM < 0.3 (↑) | avg. max TM (↓) | pairwise TM (↓) | MaxCluster (↑) |
| (no scaling) | 0.104 ± 0.019 | 0.012 ± 0.007 | 0.427 ± 0.022 | 0.197 | 0.571 |
| (scaling 2) | 0.148 ± 0.023 | 0.040 ± 0.012 | 0.403 ± 0.024 | 0.198 | 0.549 |
| (scaling 5) | 0.832 ± 0.024 | 0.312 ± 0.029 | 0.375 ± 0.011 | 0.193 | 0.387 |
| (scaling 10) | 0.952 ± 0.014 | 0.384 ± 0.031 | 0.383 ± 0.011 | 0.194 | 0.387 |
| (scaling 15) | 0.928 ± 0.016 | 0.308 ± 0.029 | 0.388 ± 0.010 | 0.199 | 0.358 |
| (scaling 20) | 0.944 ± 0.015 | 0.304 ± 0.029 | 0.395 ± 0.010 | 0.199 | 0.347 |

TABLE 18
Speed of the integration on rotations. Integrating with a faster
time for rotations compared to translation leads to more designable
structures. Reporting the mean ± std. on 278 test samples.

| Rotation time scaling | RMSD (↓) |
|---|---|
| Scale 2 | 3.641 ± 4.457 |
| Scale 5 | 3.257 ± 4.113 |
| Scale 10 | 3.334 ± 4.325 |
| Scale 15 | 3.237 ± 4.145 |

TABLE 19
Comparison of Designability (fraction with scRMSD < 2.0 Å), Novelty (max. TM-score to
PDB and fraction of proteins with averaged max. TMscore < 0.3 and scRMSD < 2.0 Å), and
Diversity (avg. pairwise TMscore and MaxCluster fraction) for different number of Euler steps at inference.

| | | Novelty | | | |
|---|---|---|---|---|---|
| | Designability | | avg. max TM | Diversity | |
| | Frac. < 2 Å (↑) | Frac. TM < 0.3 (↑) | (↓) | pairwise TM (↓) | MaxCluster (↑) |
| (15 Euler steps) | 0.480 ± 0.032 | 0.136 ± 0.022 | 0.382 ± 0.012 | 0.196 | 0.430 |
| (20 Euler steps) | 0.876 ± 0.021 | 0.328 ± 0.030 | 0.358 ± 0.009 | 0.203 | 0.341 |
| (30 Euler steps) | 0.984 ± 0.008 | 0.328 ± 0.030 | 0.385 ± 0.009 | 0.206 | 0.333 |
| (50 Euler steps) | 0.952 ± 0.014 | 0.384 ± 0.031 | 0.383 ± 0.011 | 0.194 | 0.387 |
| (75 Euler steps) | 0.800 ± 0.025 | 0.300 ± 0.029 | 0.380 ± 0.010 | 0.189 | 0.434 |
| (100 Euler steps) | 0.748 ± 0.028 | 0.340 ± 0.030 | 0.366 ± 0.011 | 0.188 | 0.415 |
| (150 Euler steps) | 0.620 ± 0.031 | 0.180 ± 0.024 | 0.408 ± 0.013 | 0.185 | 0.437 |
| (200 Euler steps) | 0.580 ± 0.031 | 0.208 ± 0.026 | 0.391 ± 0.012 | 0.183 | 0.447 |

TABLE 20
Effect of the number of integration steps on the aligned
RMSD between the generated and ground truth backbone.
Reporting the mean ± std. on 278 test samples.

| | |
|---|---|
| # steps | RMSD (↓) |

| | | |
|---|---|---|
| 30 | steps | 3.384 ± 4.223 |
| 50 | steps | 3.334 ± 4.325 |
| 100 | steps | 3.374 ± 4.377 |
| 150 | steps | 3.405 ± 4.409 |
| 200 | steps | 3.481 ± 4.465 |

Architecture ablation. In Table 5 we seek to understand the effects of architecture and dataset on the performance across our main designability, novelty, and diversity metrics for unconditional backbone generation. Starting from FOLDFLOW++we first investigate the effect of replacing the Folding Block with a simple MLP with FOLDFLOW++ (-F. Block) and removing the sequence conditioning entirely (-ESM2). In this comparison we find that both the folding block and structure conditioning significantly improve the results. We find that the Folding block improves all metrics while the structure conditioning improves designability and diversity at the cost of novelty.

Dataset and Training. In Table 5, we look at how adding synthetic data or stochastic flow matching affects designability, diversity, and novelty metrics. We find that both these additions actually hurt these metrics, although this is likely due to the change in composition of their generated structures. Overall, we find that these two models create more diverse proteins.

Number of inference steps. We also studied the influence of number of Euler steps on the generated proteins. The results are shown in Table 19. The best performance is reached for a number of steps around 50 where 50 gives the best results in terms of novelty. There is a trade-off between these results, where the novelty and diversity metric can be down to the benefits of designability.

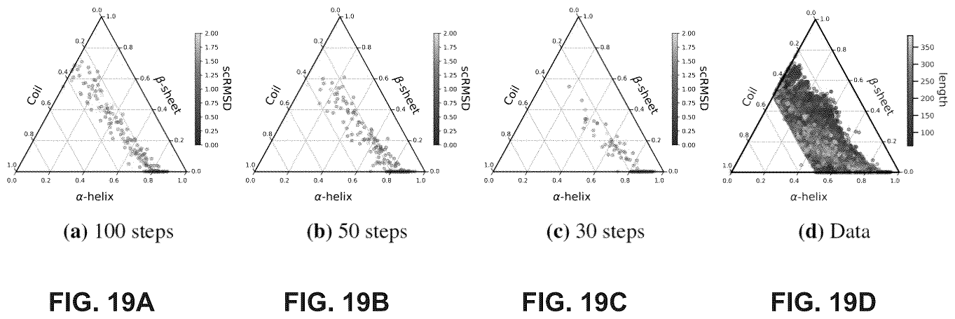

Impact of synthetic augmented dataset on diversity. One of the interesting benefits of using our synthetic augmented dataset is that it increases diversity among designable generated data. Proteins have different secondary structures such as helices, beta-sheets and coil. While our model generates a lot of helices, it is important for drug discovery applications to generate other secondary structure such as beta-sheets. As shown in FIGS. 19A-19D, we can see that our synthetic augmented dataset leads to an increased of beta-sheets.

1. A method for modeling a protein backbone structure, the method comprising:
inputting a sequence and a structure of a protein into an encoder to generate a plurality of structure representations and a plurality of sequence representations;
fusing one or more structure representations and one or more sequence representations to generate at least a joint single representation and a joint pair representation; and
inputting the joint single representation and the joint pair representation into a decoder to generate a prediction useful for modeling the protein backbone structure.

2. The method of claim 1, wherein inputting the sequence and the structure of the protein into the encoder comprises parameterizing the sequence and a backbone of the protein.

3. The method of claim 1, wherein inputting the sequence and the structure of the protein into the encoder further generates a rigid representation.

4. The method of claim 3, wherein the rigid representation includes a special Euclidean group SE(3) representing a group of rigid body motions or transformations in three-dimensional space.

5. The method of claim 1, wherein the encoder comprises a structure encoder and a sequence encoder.

6. The method of claim 5, wherein the sequence encoder comprises a protein language model.

7. The method of claim 5, wherein the structure encoder comprises an invariant point attention (IPA) transformer architecture.

8. The method of claim 1, wherein the fusing of one or more structure representations and one or more sequence representations is performed using a multi-modal fusion trunk which combines multi-modal representations of encoded structure representations and sequence representations.

9. The method of claim 8, wherein the decoder consumes the joint single representation and the joint pair representation from the multi-modal fusion trunk and outputs the prediction useful for modeling the protein backbone structure.

10. The method of claim 1, wherein one or more skip connections are present between the encoder and the decoder.

11. The method of claim 1, wherein the encoder and the decoder are structured within a generative prediction model.

12. The method of claim 11, wherein the generative prediction model further comprises a multi-modal fusion trunk.

13. The method of claim 11, wherein the generative prediction model uses flow matching comprising probability paths on SO(3) and/or matching vector fields on SO(3).

14. The method of claim 11, wherein the generative predictive model uses an SE(3)^{N}-invariant density using a flow-matching objective.

15. The method of claim 13, wherein flow matching comprises building flows on a group of rotations SO(3) and translation R³.

16. The method of claim 11, wherein the generative predictive model is trained using a loss function that decomposes into per residue rotation and translation.

17. The method of claim 11, wherein the generative predictive model is trained by curating and/or filtering datasets of protein structure using one or more of steps of:
d) filtering low-confidence structures using per-residue local confidence metrics to filter out low-confidence structures;
e) masking low-confidence residues; and
f) filter high-confidence, low-quality structures by learning a structure prediction model trained on structural features predictive of protein quality.

18. The method of claim 1, further comprising using the modeled protein backbone structure for one or more of:
j) unconditional protein backbone generation;
k) increasing secondary structure diversity;
l) protein sequence folding;
m) protein structure motif scaffolding;
n) protein equilibrium conformation sampling;
o) capturing different modes of the equilibrium conformation;
p) partial structure generation by conditioning on a masked sequence;
q) de novo drug design; and
r) engineering a structure that binds a desired target protein structure and sequence pair.

19. The method of claim 1, wherein the generated prediction comprises SE(3)^{N} vector fields.

20. The method of claim 19, further comprising modeling the protein backbone structure using the SE(3)^{N} vector fields by iteratively refining a backbone structure by applying the SE(3)^{N} vector fields to modify positions and orientations of atoms of the backbone structure.
